(19) United States
(12) Patent Application Publication
(10) Pub. No.: US 2024/0384225 A1
(43) Pub. Date: Nov. 21, 2024

(54) SUBSTRATES, SYSTEMS AND METHODS FOR CULTIVATION OF PYROPIA SPECIES

(71) Applicant: W. L. Gore & Associates, Inc., Newark, DE (US)

(72) Inventor: Norman E. Clough, Landenberg, PA (US)

(21) Appl. No.: 18/665,476

(22) Filed: May 15, 2024

Related U.S. Application Data
(60) Provisional application No. 63/466,847, filed on May 16, 2023.
(60) Provisional application No. 63/472,043, filed on Jun. 9, 2023.

(51) Int. Cl. C12N 1/12

(52) U.S. Cl. CPC C12N 1/12; C12N 2531/00; C12N 2533/30

(57) ABSTRACT

Some aspects of the disclosure relate to structures, systems, and methods for cultivating marine macroalgae (seaweed), specifically the cultivation of the macroalgae genus Pyropia. The structures, systems and methods disclosed herein provide for increased seeding density, growth rate and yield of cultivated Pyropia.

### CROSS-REFERENCE TO RELATED APPLICATION

This application claims the benefit of Provisional Application No. 63/466,847, filed May 16, 2023, and also claims the benefit of Provisional Application No. 63/472,043, filed Jun. 9, 2023, which are incorporated herein by reference in their entireties for all purposes.

### FIELD

The present disclosure relates generally to apparatuses, systems, and methods for cultivating marine macroalgae (seaweed). More specifically, the disclosure relates to apparatuses, systems, and methods for promoting the spore attachment and growth of the macroalgae genus Pyropia.

### BACKGROUND

One of the most valuable maricultured seaweed in the world is the red algae, genus Pyropia (nori). Commercial production of Pyropia has an annual value over 1 billion (US dollars) and the demand for commercial production continues to grow due to the increased use of Pyropia in human food and animal feed products. However, Pyropia also has a complex life-cycle, which creates a labor intensive, multi-step cultivation process.

As shown in FIG. 1, Pyropia spores (conchocelis) are extracted and settled, under very low light, in large stagnant tanks containing oyster shells. The spore germinates into a microscopic shell-boring conchocelis phase. The conchocelis are then stimulated to mature, by changing conditions (temperature, nutrients, aeration, etc.) to become conchospores. The conchospores are then released into the water to create a Pyropia-cultivation environment and settle (seeded) onto cultivation seedstrings or nets. In more industrialized production, nets are dipped into the tanks on a large rotary wheel or conveyer belt. The seeded needs are then placed in a nursery environment and monitored for blade (thalli) growth. Once the Pyropia thalli are of sufficient size, the nets are then outplanted to sheltered coastal areas on rafts. In seaweed farming areas of the north Eastern hemisphere, the cultivation nets are typically seeded in the fall and outplanted in late November or early December.

After 1-2 months, new adults have grown on the nets. The adult Pyropia growth from the seeded conchospores will then form archeospores. The archeospores are released into the water surrounding the adult Pyropia. Archeospores captured by the cultivation net will grow into new adult Pyropia. These nets are then lifted out of the water and most of the adult biomass is harvested, either manually or mechanically. The nets are then returned to the water to allow for the next generation of captured archeopores to grow into new adult Pyropia. The same nets are harvested multiple times during a growing season. The Pyropia biomass harvested early in the growing season primarily originate from the seeded conchospores, while the Pyropia biomass harvested later in the growing season primarily originate from captured archeospores.

While Pyropia cultivation has been ongoing for centuries, there are many areas of improvement needed in the commercial cultivation setting. Specifically, improvements in the seeding stage, the initial thalli growth, the archespore capture and growth and the final biomass harvested are all needed. The present invention addresses each of these issues.

### SUMMARY

Various embodiments are directed to the substrates and methods for cultivation of macroalgae genus Pyropia. Preferred embodiments described herein utilize a substrate composed of a microporous expanded polymer material having a surface roughness (Ra) greater than 1.0. Preferred embodiment described herein also include a substrate combining a microporous expanded polymer material having a surface roughness (Ra) greater than 1.0. and a non-porous material wherein the non-porous material is wrapped around a core of microporous expanded polymer material.

A first aspect of the invention is a seaweed cultivation medium comprising: a substrate composed of a microporous expanded polymer material and a non-porous material wherein the non-porous material is twisted around the microporous expanded polymer material, wherein the substrate is exposed to a Pyropia-cultivation environment including seawater and Pyropia conchospores dispersed in the seawater to permit seeding onto the substrate, the Pyropia-cultivation environment including an aeration system and exposure to a seawater temperature of approximately 20 degrees Celsius and photosynthetically active radiation, and wherein after 5 to 15 days of exposure to the Pyropia-cultivation environment, a surface of the submerged substrate supports a gametophyte density of 15-35 gametophytes per square millimeter.

A second aspect of the invention is a seaweed cultivation medium wherein the microporous expanded polymer material is expanded polyethylene (ePE) or expanded polytetrafluoroethylene (ePTFE).

A third aspect of the invention is a seaweed cultivation medium wherein the microporous expanded polymer material has a surface roughness (Ra) greater than 1.0.

A fourth aspect of the invention is a seaweed cultivation medium wherein the non-porous material is selected from the group consisting of polyester, polypropylene, polyamide, and polyethylene.

A fifth aspect of the invention is a seaweed cultivation medium wherein the microporous expanded polymer material exhibits no hairlike fibers protruding from the surface of the expanded polymer material

A sixth aspect of the invention is a seaweed cultivation medium wherein the substrate is exposed to the Pyropia-cultivation environment through periodic or continuous submersion of the substrate into the Pyropia-cultivation environment.

A seventh aspect of the invention is a seaweed cultivation medium wherein the substrate is exposed to the Pyropia cultivation environment less than 15 days.

An eighth aspect of the invention is a seaweed cultivation medium wherein the Pyropia conchospores are provided to the Pyropia-cultivation environment through oyster shells with conchocelis of Pyropia.

A ninth aspect of the invention is a seaweed cultivation medium, wherein the Pyropia-cultivation environment comprises a ratio of about 2 oyster shells per liter of seawater.

A tenth aspect of the invention is a seaweed cultivation medium wherein the Pyropia-cultivation environment comprises a ratio of less than 2 oyster shells per liter of seawater.

An eleventh aspect of the invention is a seaweed cultivation medium wherein the substrate has a growth potential value greater than or equal to 200, wherein the growth rate potential is determined by multiplying the average seeding density with the average germling growth rate and then by the average biomass yield for the substrate.

A twelfth aspect of the invention is a seaweed cultivation medium wherein the substrate has an overall evaluation score greater than or equal to 1.5, wherein the overall evaluation score is determined by summing the (seeding density value/highest value) and the (germling growth rate/highest growth rate) and the (biomass yield/highest biomass yield).

A thirteenth aspect of the invention is a seaweed growth medium comprising: a substrate composed of an microporous expanded polymer material having a surface roughness (Ra) greater than 1.0, wherein the substrate is exposed to a Pyropia-cultivation environment including seawater and Pyropia conchospores dispersed in the seawater to permit seeding onto the substrate, and wherein 30 days after seeding, the substrate supports juvenile Pyropia plants having an average blade size of 8-11 square millimeters.

A fourteenth aspect of the invention is a seaweed growth medium wherein the Pyropia exhibits an average growth rate of 0.25-0.35 mm²/day during the 30 days after seeding.

A fifteenth aspect of the invention is a seaweed growth medium comprising: a substrate composed of a microporous expanded polymer material and a non-porous material wherein the non-porous material is twisted around the microporous expanded polymer material, wherein the substrate is disposed in a Pyropia-cultivation environment including seawater and Pyropia spores dispersed in the seawater to permit seeding onto the submerged substrate, and wherein 60 days after seeding, a surface of the submerged substrate supports an average Pyropia biomass yield of 6-10 mg/mm², wherein the surface seawater was removed from the Pyropia biomass prior to weighing.

A sixteenth aspect of the invention is a seaweed growth medium comprising: a substrate composed of a microporous expanded polymer material and a non-porous material wherein the non-porous material is twisted around the microporous expanded polymer material, wherein the microporous expanded polymer material having a surface roughness (Ra) greater than 1.0, and wherein the substrate is exposed to a Pyropia-cultivation environment including seawater and Pyropia conchospores dispersed in the seawater to permit seeding onto the substrate, the Pyropia-cultivation environment including an aeration system and exposure to a seawater temperature of approximately 20 degrees Celsius and photosynthetically active radiation, and wherein after 5 to 15 days of exposure to the Pyropia-cultivation environment, a surface of the submerged substrate supports a gametophyte density of 15-35 gametophytes per square millimeter, and wherein 30 days after seeding, the substrate supports juvenile Pyropia plants having an average blade size of 8-11 square millimeters, and wherein 60 days after seeding, a surface of the submerged substrate supports an average Pyropia biomass yield of 6-10 mg/mm², wherein surface seawater was removed from the Pyropia biomass prior to weighing.

### BRIEF DESCRIPTION OF THE FIGURES

FIG. 1. An illustration of the commercial Pyropia cultivation cycle.

FIG. 2A and 2B. Scanning Electron Micrograph (SEM) images of seaweed cultivation substrate A at low and high magnifications, respectively. The images are to the scale noted in the figure.

FIG. 2C and 2D. Scanning Electron Micrograph (SEM) images of seaweed cultivation substrate B at low and high magnifications, respectively. The images are to the scale noted in the figure.

FIG. 2E and 2F. Scanning Electron Micrograph (SEM) images of seaweed cultivation substrate C at low and high magnifications, respectively. The images are to the scale noted in the figure.

FIG. 3A-G. Photographs of Neopyropia yezoensis germlings on seven substrates after 15 days. The images are to the scale noted in the figure. FIG. 3A: Substrate JP1, FIG. 3B: Substrate JP2, FIG. 3C: Substrate CN, FIG. 3D: Substrate KR, FIG. 3E: Substrate A, FIG. 3F: Substrate B, FIG. 3G: Substrate C.

FIG. 4. Bar graph illustrating seeding density on unit area of substrate. The seeding density is provided as number of attached gametophytes per mm². Values are expressed as mean±standard deviation (n=3).

FIG. 5A-G. Photographic image of N. yezoensis blades used for growth rate measurement after 1 month for each substrate. The images are to the scale noted in the figure. FIG. 5A: Substrate JP1, FIG. 5B: Substrate JP2, FIG. 5C: Substrate CN, FIG. 5D: Substrate KR, FIG. 5E: Substrate A, FIG. 5F: Substrate B, FIG. 5G: Substrate C.

FIG. 6. Bar graph illustrating the growth rate of N. yezoensis on each substrate. Growth rate is provided as blade area (mm²) of twenty attached blades on each substrate after one month. Values are expressed as mean±standard deviation (n=3).

FIG. 7. Bar graph illustrating the yield of N. yezoensis on each substrate. Yield is provided as mg of thallus per unit area of substrate. Values are expressed as mean±standard deviation (n=3).

FIG. 8A-J. Scanning Electron Micrograph (SEM) images of N. yezoensis holdfast attachment on each substrate. The images are to the scale noted in the figure. FIG. 8A: Substrate JP1, FIG. 8B: Substrate JP2, FIG. 8C: Substrate CN, FIG. 8D: Substrate KR, FIG. 8E-8F: Substrate A, FIG. 8G-8H: Substrate B, FIG. 81-8J: Substrate C.

FIG. 9A-C. Line graphs illustrating (A) number of blades, (B) weight, and (C) area for Pyropia yezoensis grown on a cultivation net created from Substrate C or a cultivation net created from Substrate KR, on sampling dates Dec. 13, 2023, Jan. 30, 2024, Mar. 7, 2024 and Mar. 26, 2024. Statistical significance between the two cultivation nets is designated by (*).

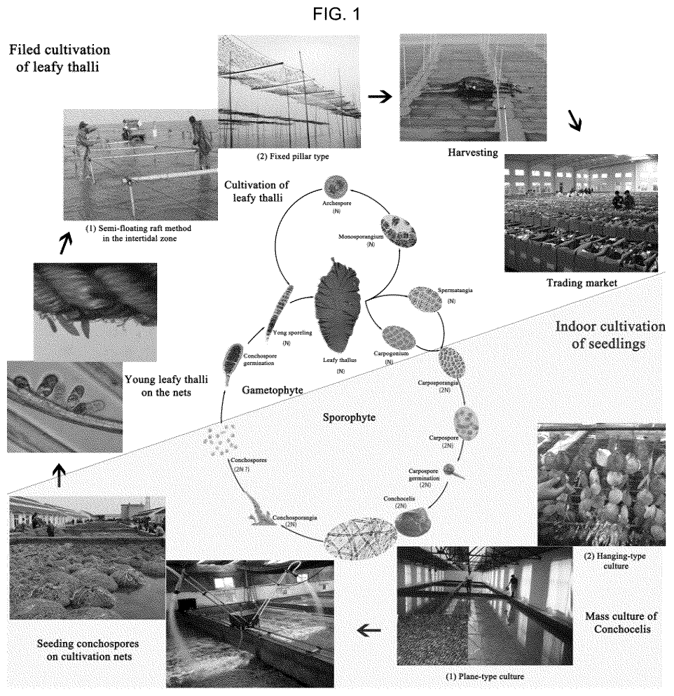

Persons skilled in the art will readily appreciate that the accompanying drawing figures referred to herein are not necessarily drawn to scale but may be exaggerated or represented schematically to illustrate various aspects of the present disclosure, and, in that regard, the drawing figures should not be construed as limiting.

### DETAILED DESCRIPTION

### Definitions and Terminology

This disclosure is not meant to be read in a restrictive manner. For example, the terminology used in the application should be read broadly in the context of the meaning to which those in the field would attribute such terminology.

The terms macroalgae and seaweed are used interchangeably throughout this disclosure. The genus Pyropia is sometimes referred to as Neopyropia and was previously referred to as Porphyra. Use of the term Pyropia in the present disclosure is intended to encompass all species attributed to this genus regardless of the variation of the naming convention used for the genus.

As used herein, the term “seedstring” is understood to mean a form of seaweed growth medium upon which seaweed spores attached and subsequently grow in seaweed mariculture. Traditionally, a seedstring may comprise two or more individual substrates, such natural fibers and/or polymer yarns or cords, twisted or wrapped together. As described herein, the composition, texture, and performance of the seedstring varies based upon the substrate materials (including but not limited to the type of polymer) used in creating the seedstring. The seedstring may then be used to create other larger cultivation structures such as ropes, braids, weaves and nets for use in large scale cultivation of macroalgae.

As used herein, the terms “seeding”, “settlement” and “gametophyte attachment” refer to and or describe the initial settlement of Pyropia conchospores onto the seaweed cultivation substrate. The terms may also be used herein to refer to or describe the subsequent settlement of Pyropia archeospores onto the seaweed cultivation substrate during the growing season.

The terms germling, thallus, and blade refer to the body of the Pyropia plant, either in its juvenile or adult state. For purposes of the present disclosure these terms are used interchangeably in reference to the growth rate and yield and the specific term used is reflective only of the stage of the development of the Pyropia plant being measured and or referenced.

A holdfast is a root-like structure at the base of a seaweed plant that fastens it to a substrate, such as a rock, for example. Holdfasts and roots differ in shape and structure between species. Root-like structures can extend from the holdfast to further fasten the seaweed to a substrate. Substrate type can also affect holdfast and root shape and structure. Seaweed holdfasts differ from the roots of land plants because the seaweed holdfast has no nutrient-absorbing function as seen with roots of land plants, but both holdfasts and roots have similar anchoring functions in land and sea plants.

With respect to terminology of inexactitude, the terms “about” and “approximately” may be used interchangeably to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

### Seeding of Seedstring and Nets for Pyropia Cultivation

Spore settlement (i.e., seeding) is a critical step in Pyropia aquaculture, as it is the initial step of the gametophyte growth and can affect the quality and production of the alga. Additionally, the second seeding of the seedstring and nets by the archeospores is critical to extending the growing season of the Pyropia aquaculture.

The present disclosures relate to improved initial and subsequent seedings of seedstring and nets used for the cultivation of Pyropia. In some embodiments of the invention, the cultivation substrate comprises a microporous expanded polymer material, such as expanded polyethylene (ePE) or expanded polytetrafluoroethylene (ePTFE), having an Ra value>1.0. The microporosity of the expanded polymer material may be due to the expansion of the polymer material and is characterized by a plurality of microscopic spaces positioned throughout the polymer material. The microscopic spaces may range in size from about 1 μm to about 200 μm. In some embodiments, the microporous expanded material may be combined, as a twist or a wrapped core with a non-porous material, such as polyester, polypropylene, polyamide, or polyethylene. In some embodiments, the cultivation substrate is in the form of a seedstring, where a non-porous substrate is twisted around a microporous substrate core to form a non-porous: microporous expanded polymer substrate. In other embodiments, the material can be a braided or twisted yarn with or without other fibers such as natural fibers or non-porous synthetic fibers such as polypropylene and polyester. All of these seedstring embodiments may be used to form cultivation nets or other woven structures for the cultivation of Pyropia.

The non-porous:microporous expanded polymer material cultivation substrates of the present invention show an increase in conchospore/seed settlement on the cultivation substrates than observed in current commercially available substrates, which include yarns of one more substrates such as polyvinyl alcohol, polyamide, polyethylene and cotton. This increase in seeding density is reflected in a higher density of Pyropia gametophytes per area on seedstring comprising the non-porous:microporous expanded polymer substrate.

A common theory in the art of Pyropia cultivation is that substrates having a greater number of individual fibers extending from the surface of the substrate (i.e., a “hairier” surface) will achieve greater seeding densities due to a higher exterior surface area available for the Pyropia conchospores to attach. Surprisingly, as disclosed herein the substrate comprising non-porous:microporous expanded polymer material where the microporous expanded polymer material has a rough surface (Ra>1.0) but no individual fibers extending from the surface exhibited a higher seeding density than those exhibited by the commercial cultivation substrates having a “hairier” surface, such substrate CN, composed of cotton and polyethylene. Notably, while substrate CN achieved a seeding density slightly above many of the other substates tested (though not as high as the non-porous:microporous expanded polymer substrate disclosed herein), it exhibited the lowest growth rate and yield of the Pyropia plants due to the weak rhizoid attachment of Pyropia to the individual fibers of the CN substrate.

The higher conchospore seeding density obtained through the cultivation substrates of the present invention allow for a reduction in the time needed for the initial seeding of the cultivation substrate to achieve Pyropia seeding densities at or above current commercially acceptable levels. Through the higher seeding density achievable with the non-porous:microporous expanded polymer substrate, the substrate may be exposed to the Pyropia-cultivation environment for a shorter period of time to achieve the desired conchospore seeding density. Additionally, and/or alternatively, the higher seeding density obtained through the cultivation substrate of the present invention comprising the non-porous:microporous expanded polymer material allows for a decreased ratio of oyster shells per liter of seawater to create the Pyropia-cultivation environment sufficient to achieve a seeding density at or above current commercially acceptable levels.

### Germling and Thallus Growth

In the nursery, the seeded cultivation nets are raised out of the water daily to expose the juvenile Pyropia plants (thallus) to air and sun. This is necessary to reduce fouling (e.g., growth of other seaweed species or microscopic algae such as diatoms) of the cultivation nets. A rapid initial growth rate of the juvenile plants is important for the young plants to compete with foreign plants and organisms and reduce the likelihood of fouling.

Once blades are 2-3 mm in length, the nets can be transferred to the farm sites or frozen for later use, as frozen nets can be used to replace lost or damaged nets during the growing season. The robust growth rate provided by the cultivation substrates of the present application allow the plants to achieve a size sufficient for out-planting to the farm sites sooner, thus reducing the nursery time needed and increasing the total time span for harvesting during the season.

Initial plant growth rate (after 30 days) was highest for the cultivation substrates composed of a microporous expanded polymer material, with an average growth rate of 0.25-0.35 mm²/day during the 30 days after seeding While the cultivation substrate composed of the non-porous:microporous expanded polymer material combination exhibited initial blade growth at or slightly below cultivation substrates composed of polyvinyl alcohol, polyamide, polyethylene and cotton, this was due to the much higher conchospore/seed density exhibited by this cultivation substrate and the plant growth ultimately exceeded those obtained by the alternative cultivation substrates.

### Thallus Yield

Out-planted Pyropia is allowed to grow to 15-30 cm in about 40-50 days before harvesting. As the thalli mature into the adult stage during this growth before and between harvests, they release archeospores into the water around the cultivation nets. After a harvest, the thalli remaining on the cultivation nets are allowed to grow and may be ready for a second harvest after another 15-20 days. Additionally, young Pyropia blades grow from archeospores captured by the cultivation net. These blades formed from the archeospores begin to contribute to the blades harvested from the cultivation nets during the second, third, fourth and later harvests. Nets may be harvested 6-8 times during a growing season (every 15-20 days). The biomass yield is one of the most important indicators for the productivity of cultivation net. The seeding density and growth rate of the Pyropia plants on the cultivation substrate affect the yield of that substrate. Furthermore, Pyropia mariculture takes place in open ocean waters. The cultivation nets may be subjected to adverse weather conditions, such as storms, which may have strong wave and/or wind conditions associated with them that can negatively impact the Pyropia. The storm conditions may not only damage the Pyropia blades but may also be strong enough to pull loosely attached Pyropia from the cultivation nets. Thus, the impact on the Pyropia harvests and financial damage to Pyropia farmers can be significant following a storm. Pyropia grown on the cultivation medium of the present application, comprising the seed string and/or cultivation nets formed from microporous expanded polymer material and a non-porous material twisted around the microporous expanded polymer material, showed increased resistance to blade loss due to storm conditions.

Total biomass achieved using the cultivation substrates comprising a microporous expanded polymer material having a rough surface texture (Ra>1.0) as described herein exceeded those achieved by the commonly used cultivation substrates composed of polyvinyl alcohol, polyamide, polyethylene and cotton. Surprisingly, the total biomass achieved with the cultivation substrate composed of the non-porous:microporous expanded polymer material combination was three times that achieved by these alternative cultivation substrates.

### Evaluation and Choice of Cultivation Substrate

Comprehensive evaluations were performed of the different substrates based on their performance in the conchospores attachment (seeding), germling growth rate and initial yield. In the first method, seven seedstrings were ranked according to their performance: seeding density, germling growth rate and yield. The score of seedstrings in each aspect was calculated by dividing that value by the highest value. Then, three scores were summed up to make the final ranking. In the second method, the seeding density was multiplied by germling growth rate and then by yield to calculate the growth potential score of each seedstring.

### EXAMPLES

### Example 1: Seeding Evaluation

A measurement of the efficiency of Pyropia conchospores attachment and germination using different seaweed cultivation substrates during early developmental stages was made using light and scanning electron microscopy (SEM). Neopyropia yezoensis, is the main species of Neopyropia in aquaculture cultivation in China, Korea and Japan; therefore, it was the species used in the following examples. However, it is understood that the results achieved would also be achieved with other species of Pyropia (Neopyropia) in use or developed for maricultivation.

Statistical analysis of the results of seeding density, growth rate and yield described in these examples was performed using graphing and analysis software, Origin® 2019 s (Origin Lab, Northampton, MA, USA). One-way analysis of variance (ANOVA) was performed to determine the significant differences among seedstrings, followed by post-hoc Tukey test. The difference was considered as statistically significant when probability (p) values of one-way ANOVA tests was smaller than 0.05.

### Substrate Compositions and Structures Tested

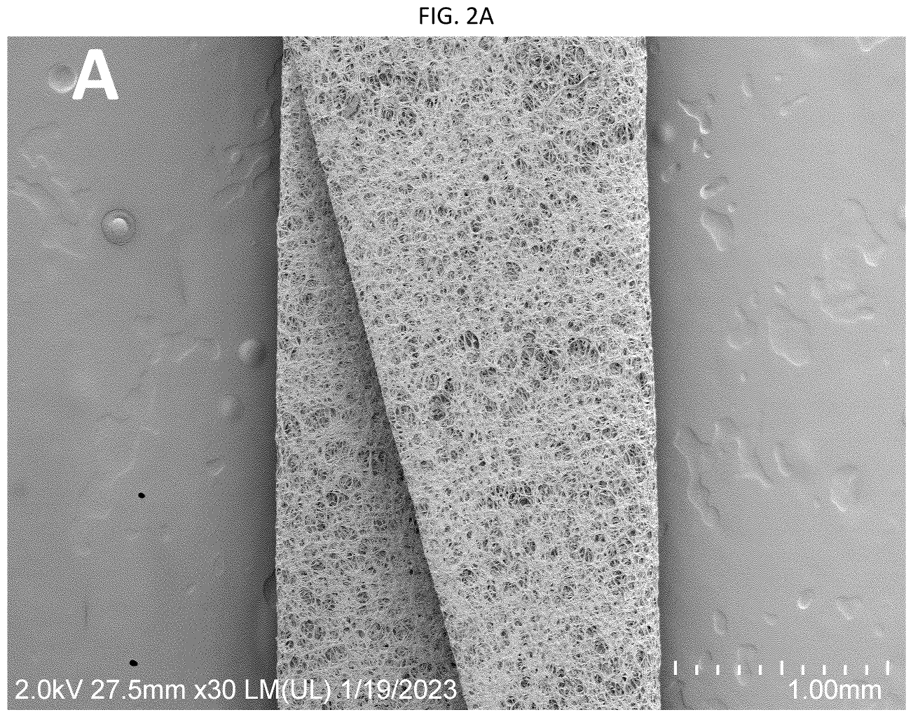

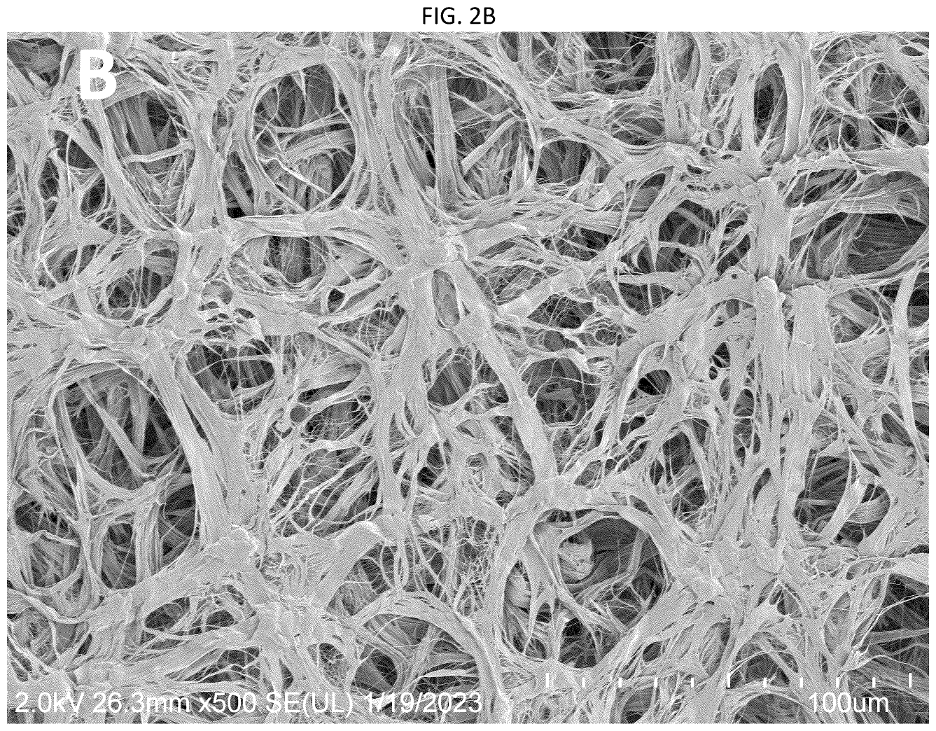

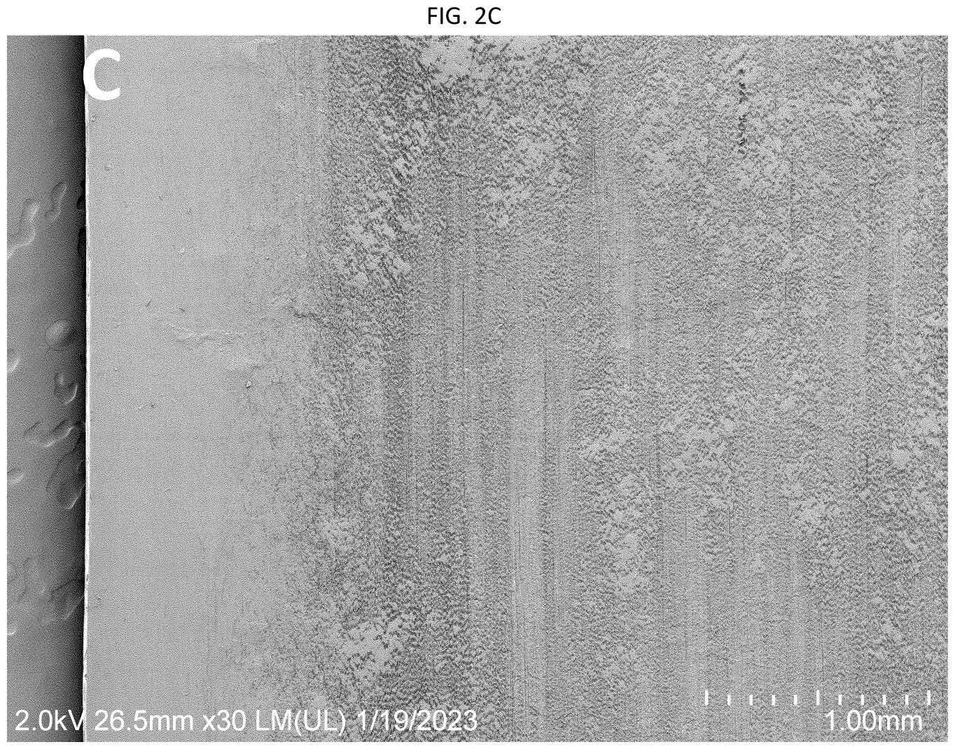

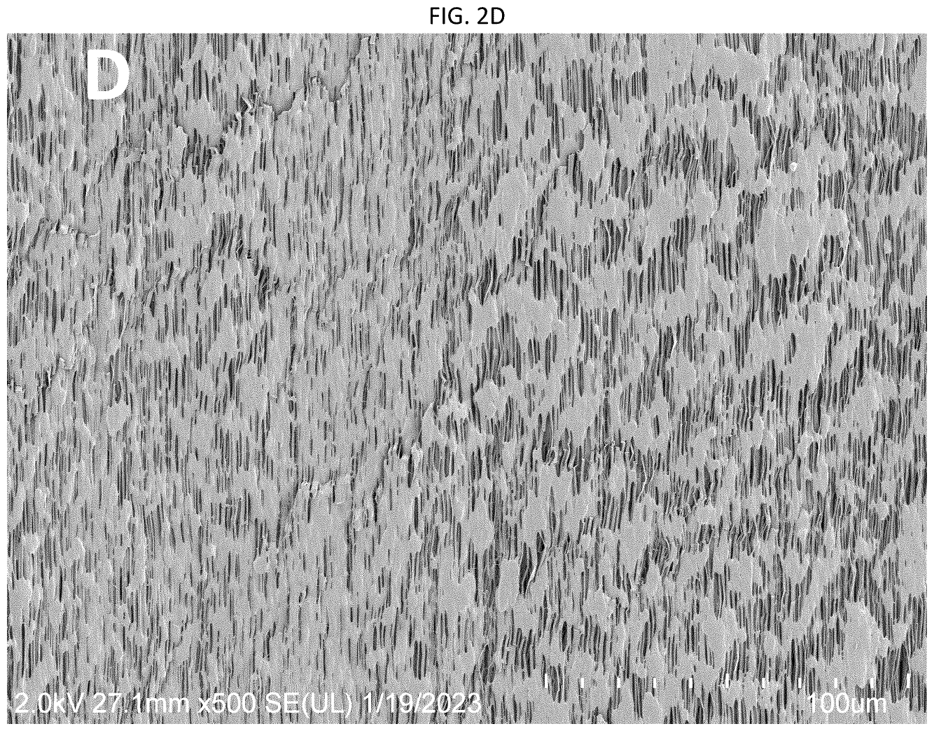

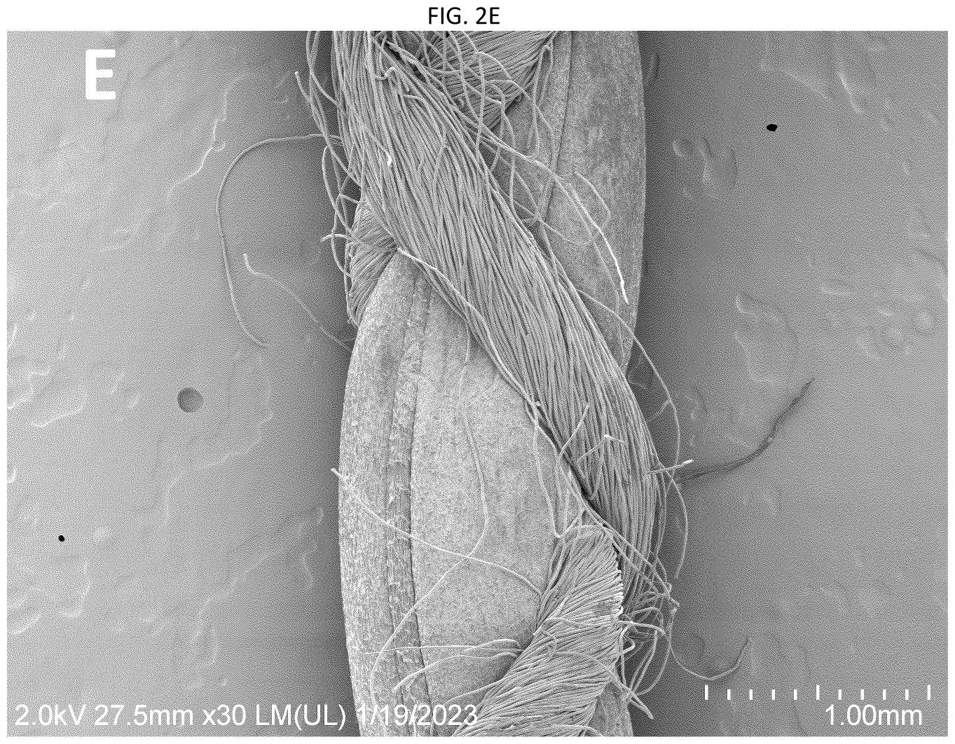

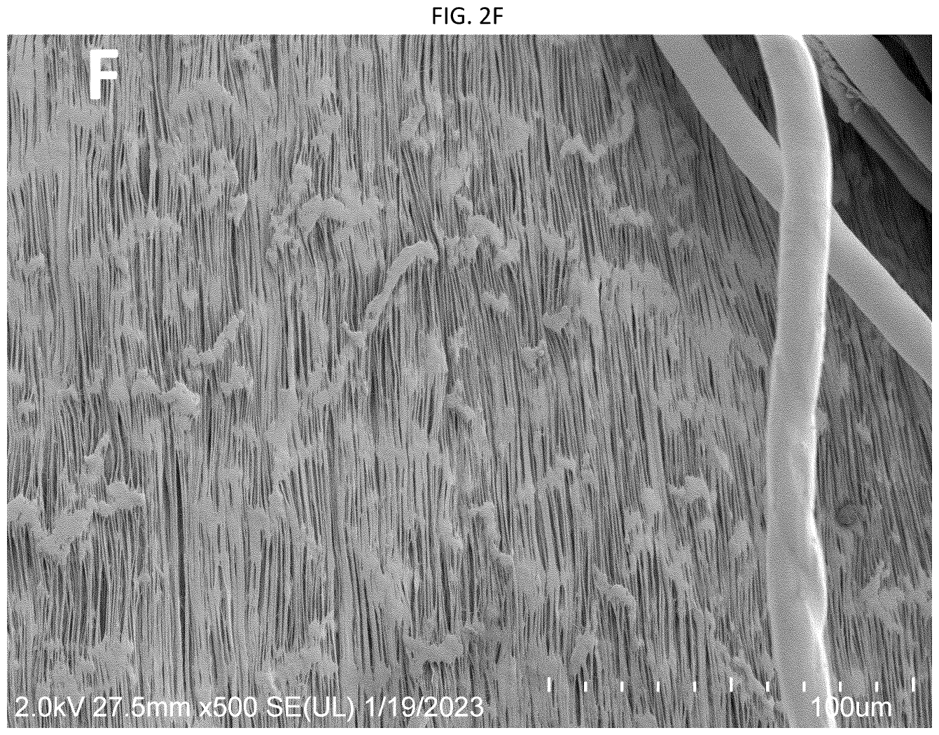

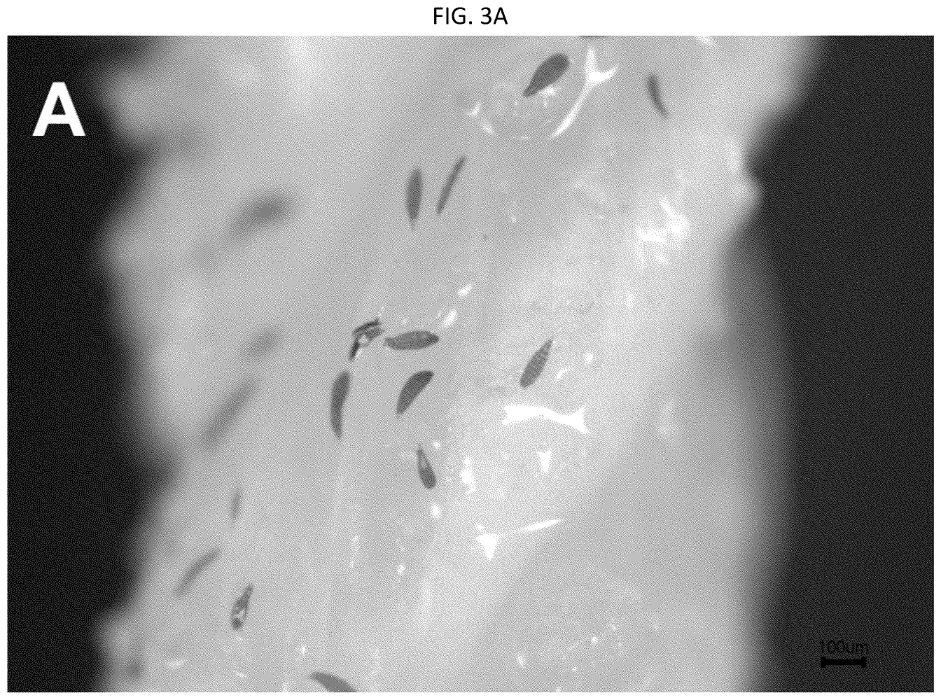

The chemical composition and texture of the seaweed cultivation substrates tested were as follows: substrates JP1 and JP2 were two Japanese commercial seedstrings made of polyvinyl alcohol (PVA) and polyamide (Nylon); substrate CN was a commercial polymer yarn seedstring from China containing cotton and polyethylene; substrate KR was a commercial seedstring from Korea containing only polyamide; substrate A was made by twisting one microporous expanded polyethylene yarn with a rough micro-structure (Ra=2.5 microns) on the surface (Gore 1266-46) (See FIG. 2A and 2B); substrate B was also a microporous polyethylene but in a tape shape and having a smooth micro-structure (Ra=<1) at the surface (Gore 12466-64-2) (See FIG. 2C and 2D); and substrate C (containing 44% by weight of polyester and 56% by weight of microporous polytetrafluoroethylene) was made by twisting polyester around a microporous polytetrafluoroethylene core (Gore R6082) with a Ra surface roughness of 1.2 to form a non-porous:microporous expanded polymer substrate (See FIG. 2E and 2F).

TABLE 1
Chemical composition and structure of each
seaweed cultivation substrate tested.

| Substrate | Chemical composition | Structure |
|---|---|---|
| JP1 | Polyvinyl alcohol and polyamide | 3 strands twisted yarn |
| JP2 | Polyvinyl alcohol and polyamide | 2 strands twisted yarn |
| CN | Cotton and polyethylene | 3 strands twisted yarn |
| KR | Polyamide | 2 strands twisted yarn |
| Gore A | Polyethylene | 1 strand twisted yarn |
| Gore B | Polyethylene | Tape |
| Gore C | Polyester and | polyester twisted around a |
| | polytetrafluoroethylene | polytetrafluoroethylene core |

### Conchospores Release and Seeding of Substrates

The oyster shells with conchocelis of the Pyropia species Neopyropia yezoensis were collected from the Aquatic Plant Variety Center, National Institute of Fisheries Science and maintained in a 20° C. culture room of the Marine Ecology and Green Aquaculture (MEGA) Laboratory, Incheon National University. These shells were cultured in the artificial seawater with von Stosch enriched seawater medium (VSE) at 40 μmol photons m−2 s−1 with a 12:12 h, L:D cycle using white LED lights. For the conchospores release and seeding, each of the seven substrates tested were wrapped around the glass slides and placed randomly in a culture vessel (n=3) with 1 L VSE artificial seawater. Oyster shells were then placed in the middle of the culture vessel with gentle aeration. A ratio of 2 oyster shells per 1 L VSE was used.

### Microscopic Observation and Seeding Density Determination

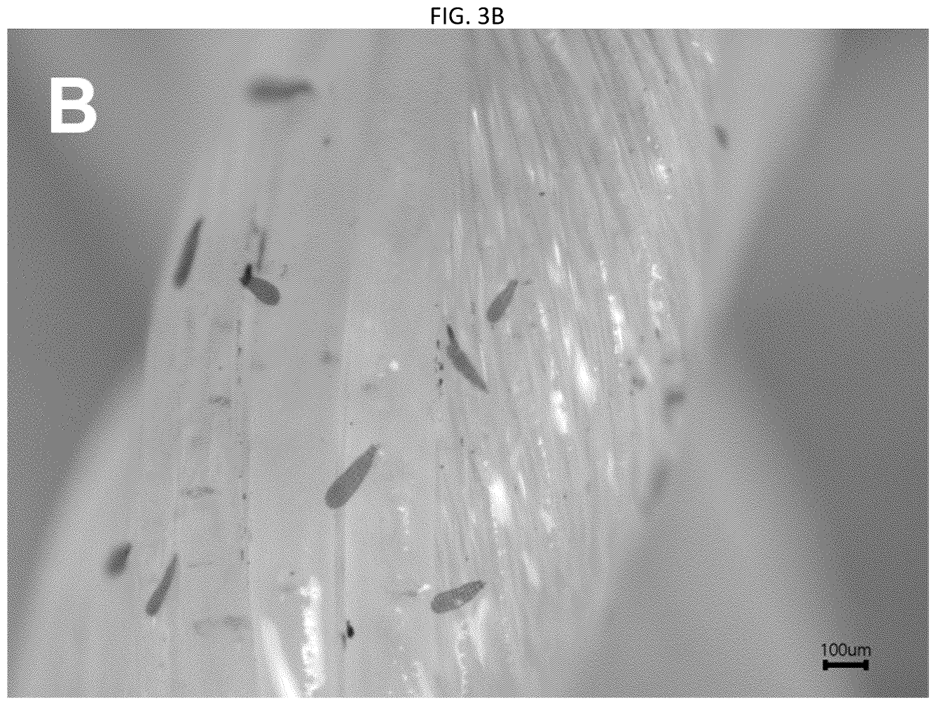

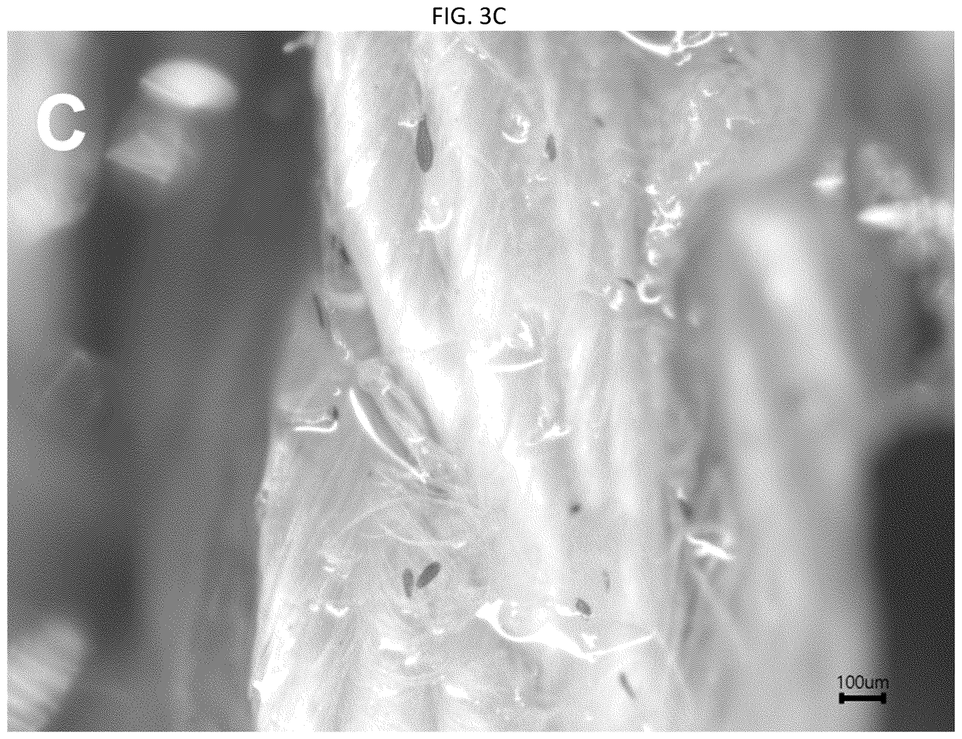

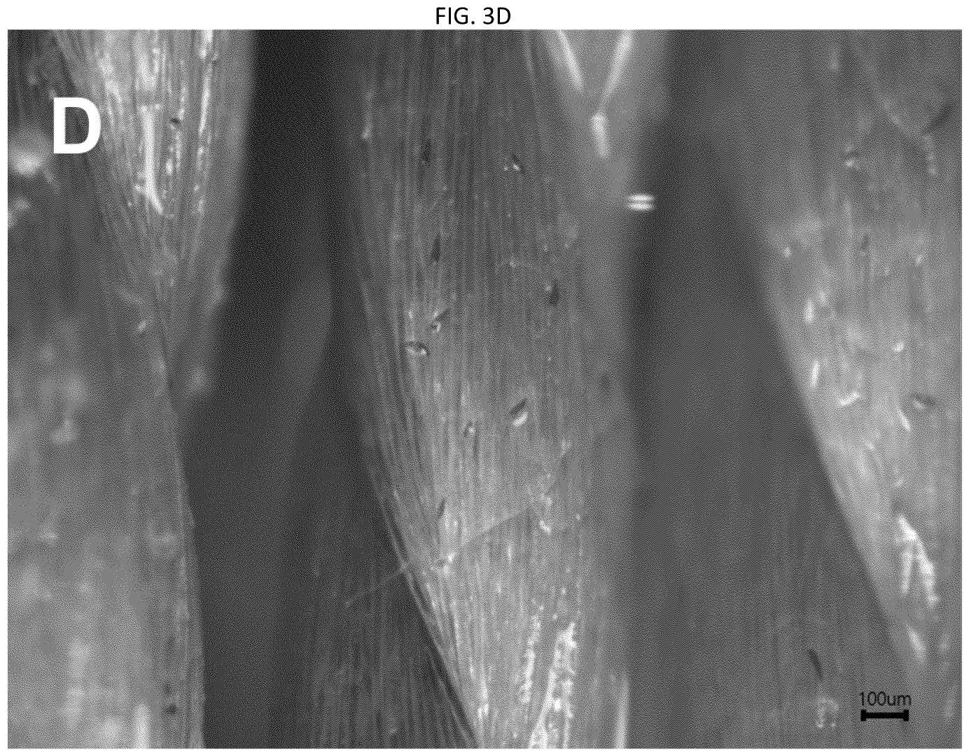

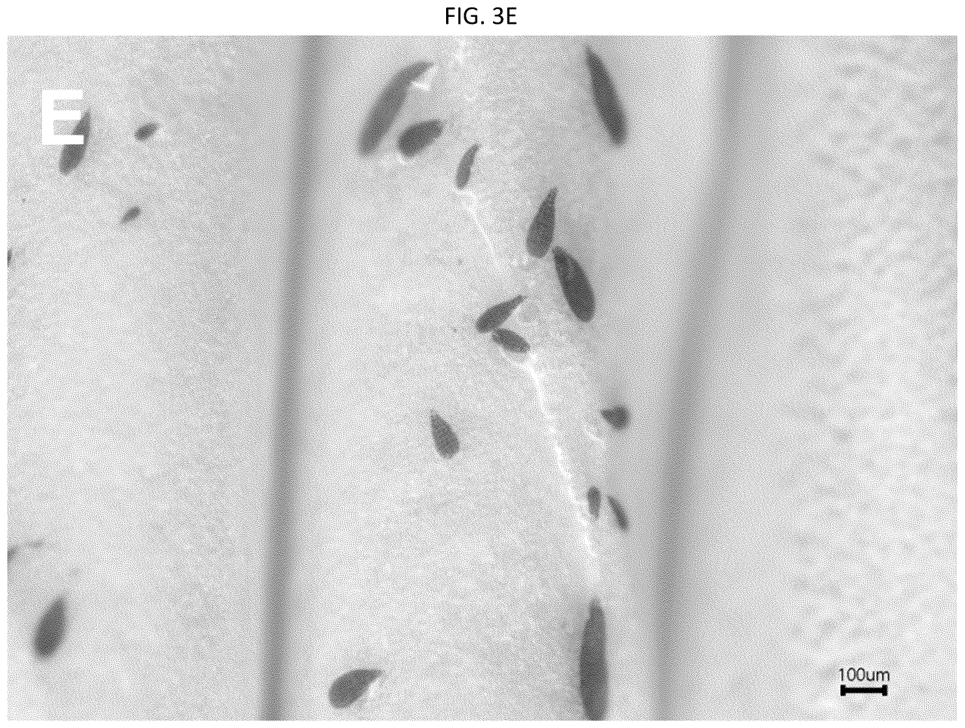

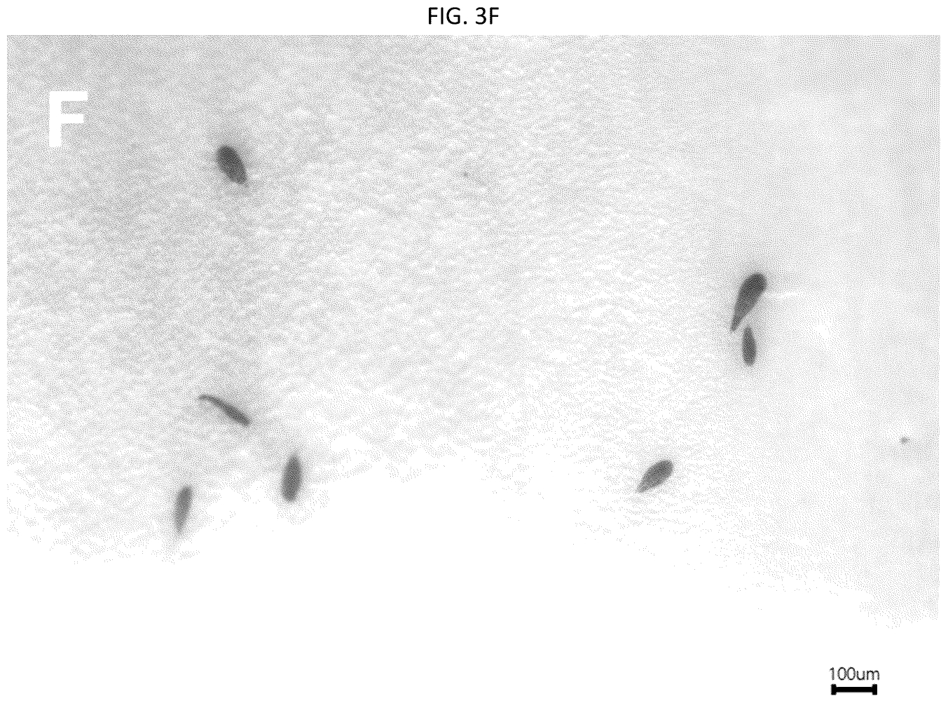

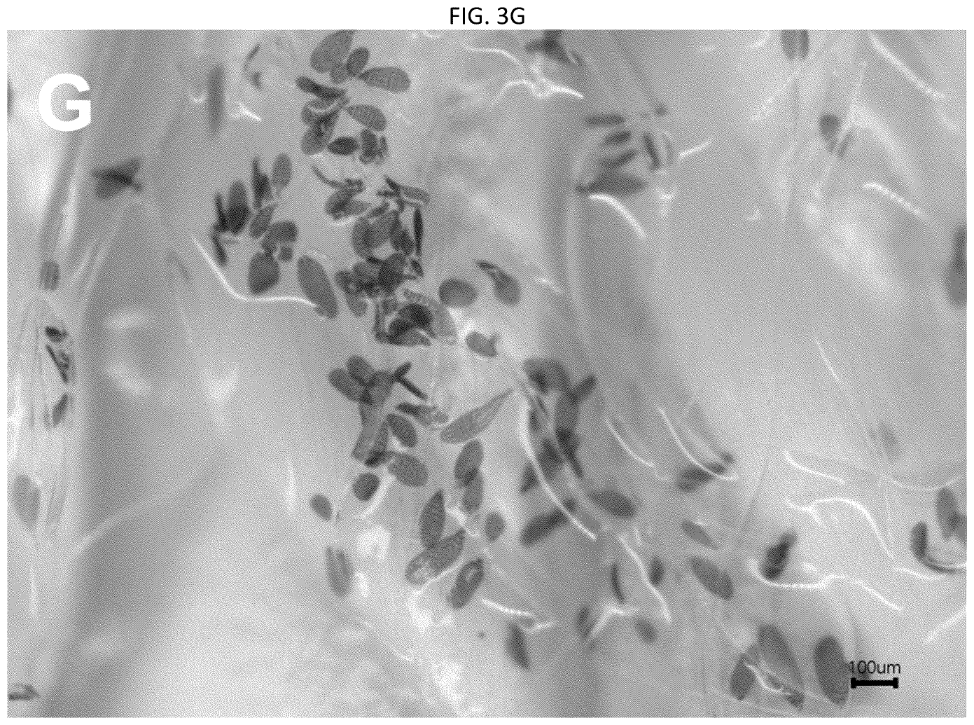

The substrates were observed using a light microscope (Nikon, Tokyo, Japan) for spore attachment and germination every 5 days for 15 days. On the 15th day, the germlings on the substrates were counted and the seeding density was measured at five random spots for each substrate (See FIG. 3A-G).

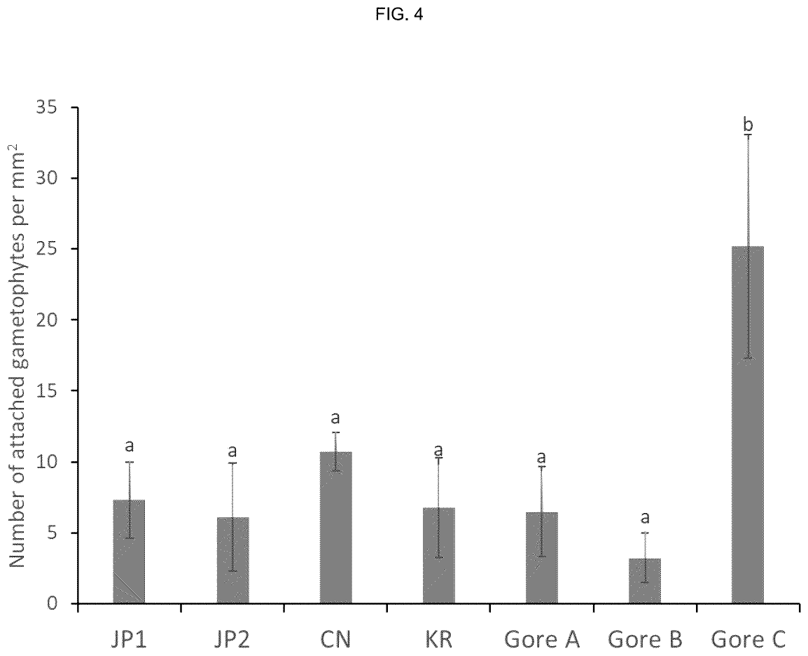

Based on the result of microscopic observation and counting, the average seeding density of substrate C was 25 spores per mm², which was much higher than other substrates (FIG. 4). The lowest seeding density was observed on substrate B (3-seeding per mm²) (FIG. 4). No significant differences were observed among substrates A, JP1, JP2, CN and KR, whose seeding densities were ranged from 6 to 10 seeding per mm² (FIG. 4).

After 15 days of cultivation, the substrates with attached germlings were transferred to 10° C. with the PAR of 100 μmol photons m−2 s−1 and a 12:12 h, L:D cycle. The substrates were then placed in 2 L culture vessels with VSE artificial seawater and aeration. The culture medium was changed every week.

### Example 2: Growth Rate and Yield Evaluation

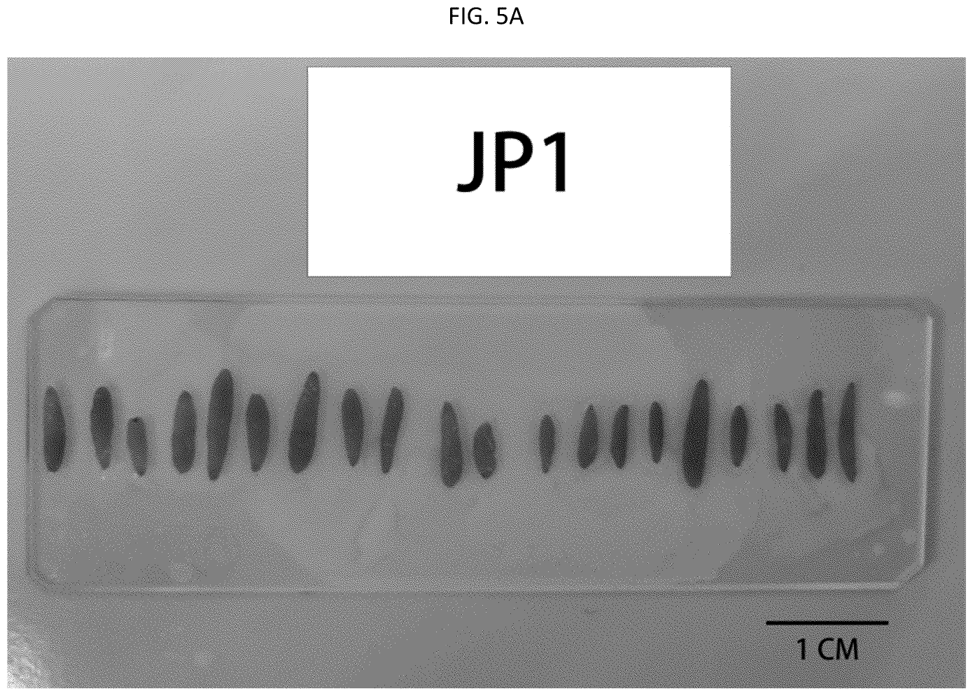

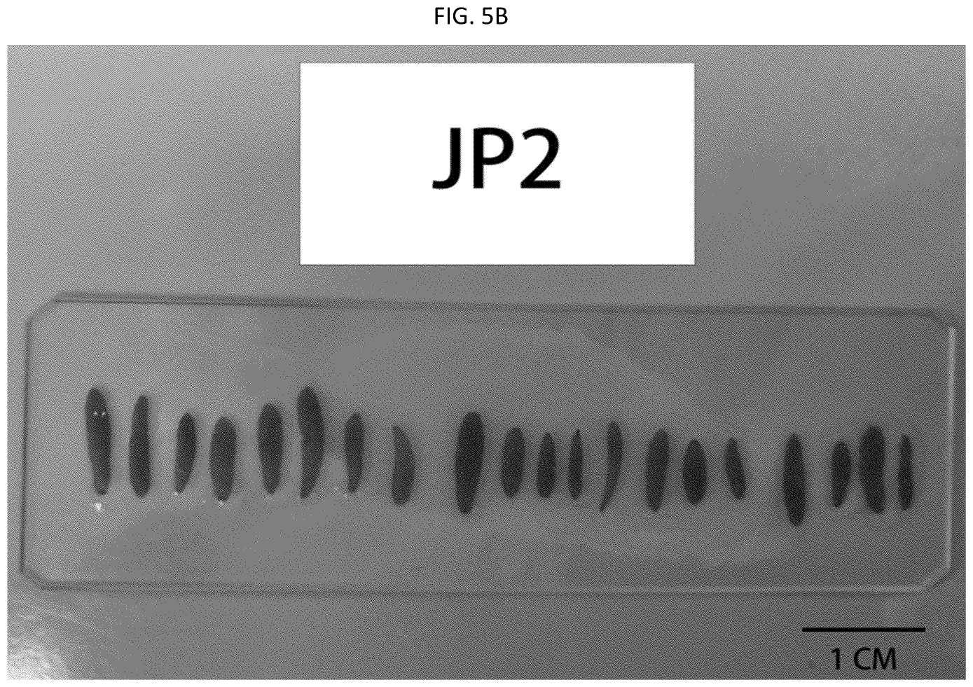

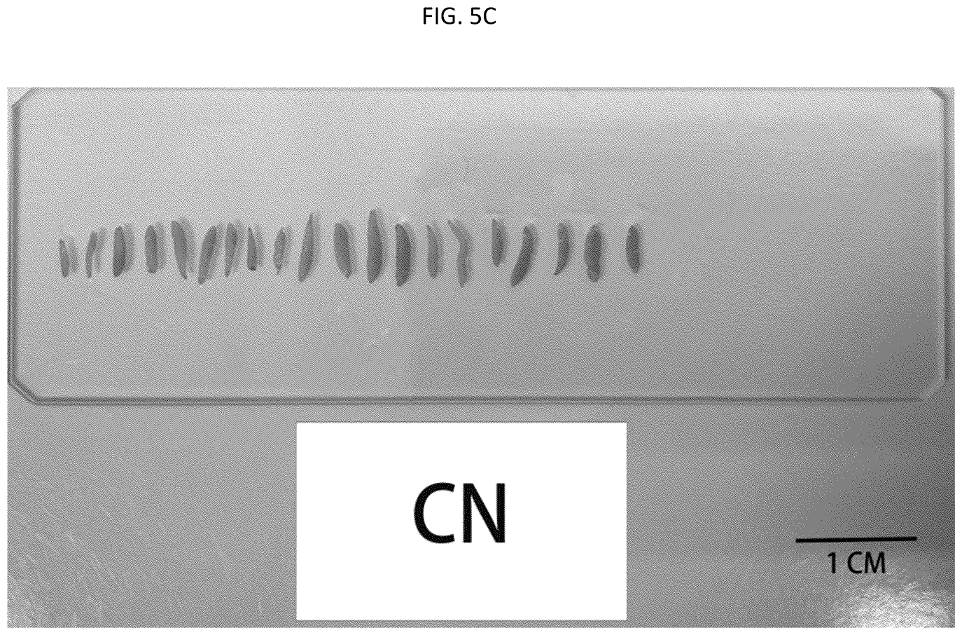

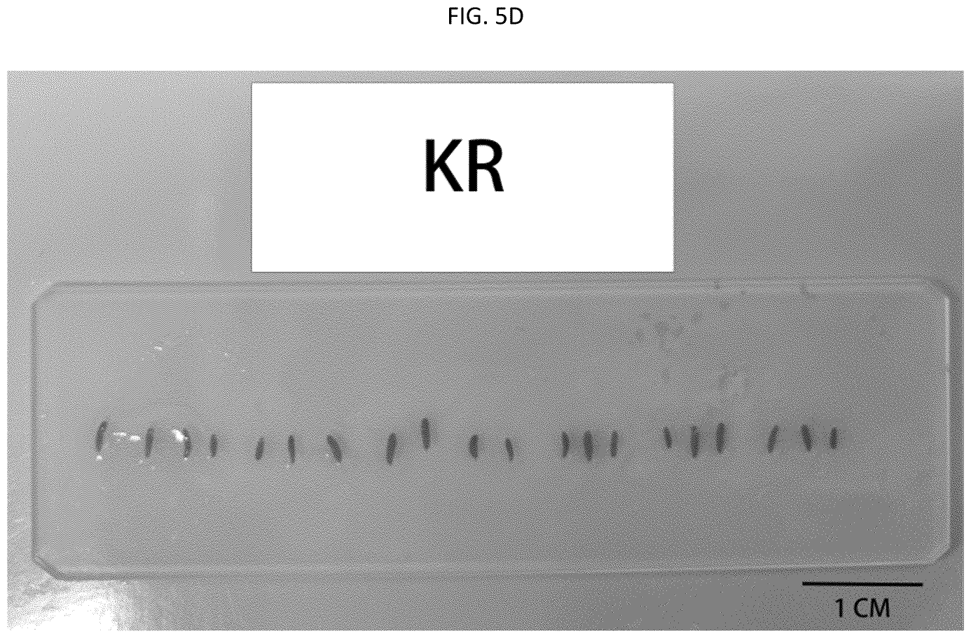

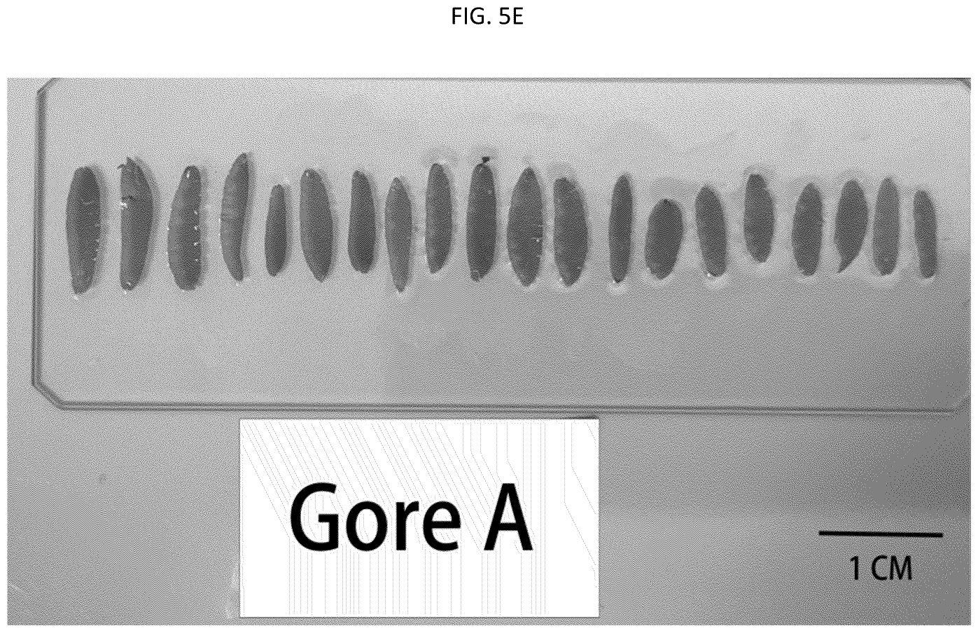

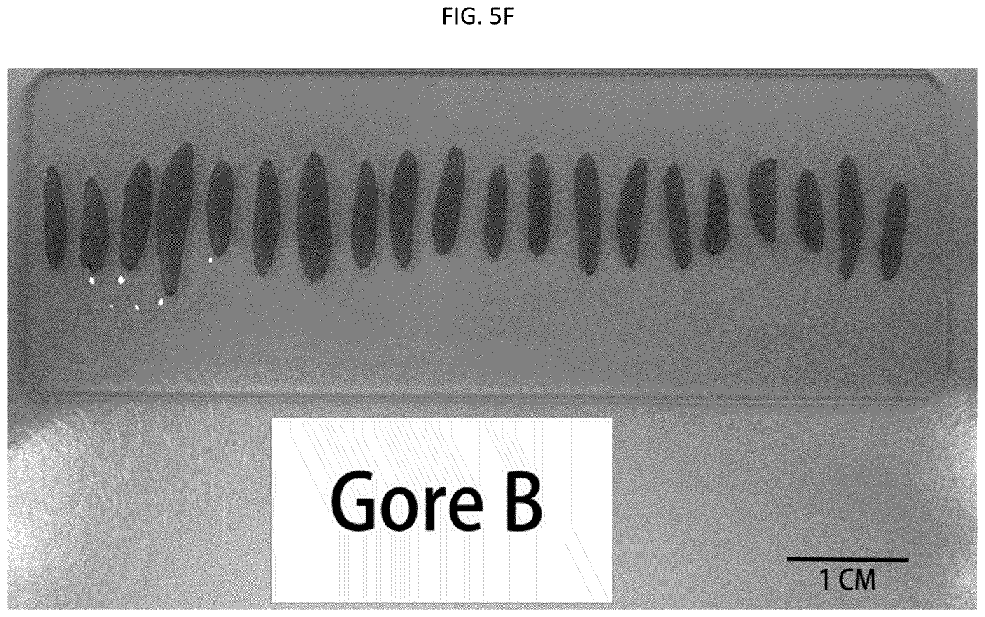

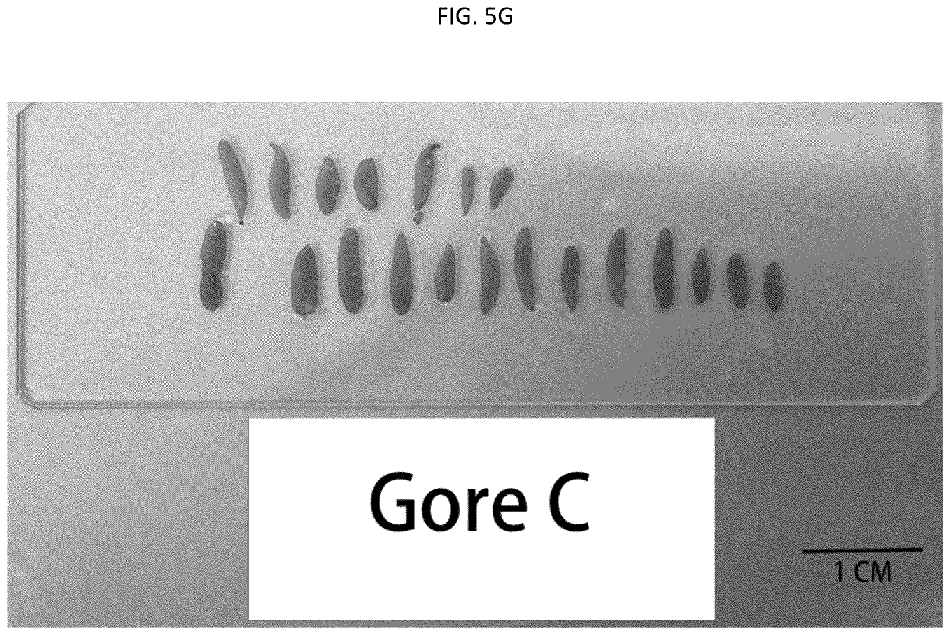

After one month, twenty juvenile Neopyropia yezoensis blades were randomly selected from each substrate (FIG. 5). The area of these blades was measured using image processing and analysis in Java (ImageJ) software (version 1.43, Synoptics, Cambridge, UK) to estimate the growth rate of juvenile N. yezoensis blades on each substrate.

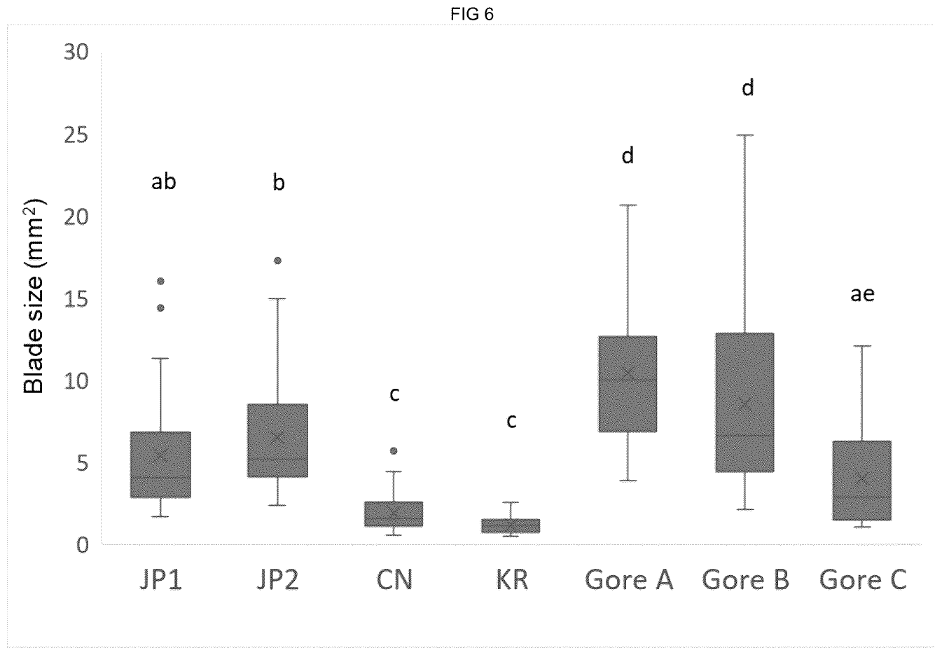

Growth rates of juvenile Neopyropia yezoensis blades were dissimilar across various substrates (FIG. 6). Substrate A showed the highest growth rate with an average blade area of 10.42 mm² (FIG. 6). Blades on substrate B had an average area of 8.53 mm², which was the second highest value among all substrates, followed by substrates JP2 and JP1 with an average blade area of 6.49 and 5.39 mm², respectively. Substrate C was ranked at the fifth place with an average blade area of 3.98 mm². Blades on Substrates CN and KR had the lowest growth rate, with average blade area of 1.88 and 1.15 mm², respectively (FIG. 6).

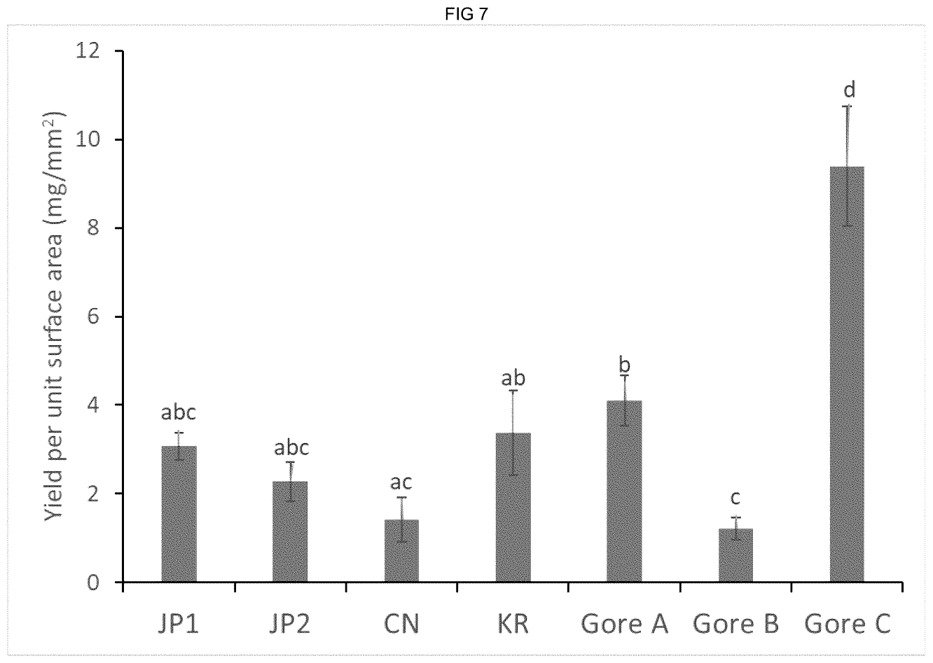

After one additional month, measurement of N. yezoensis yield was conducted. For each substrate, the thallus on 1 cm of the substrate was harvested and blotted dry with a paper towel. The thallus was weighed, and the yield on unit area was calculated by dividing the weight by the estimated surface area of the substrate. Substrate C had the best performance, with the average yield of 9.38 mg/mm² (FIG. 7), followed by the substrate A, with the average yield of 4.09 mg/mm². Substrates KR, JP1 and JP2 had similar performance, with the average yield of 3.37, 3.07 and 2.28 mg/mm², respectively (FIG. 7). Substrates CN and B had the lowest yield among all substrates, with the average yield of 1.41 and 1.21 mg/mm², respectively (FIG. 7).

### Example 3: Evaluation of N. yezoensis Attachment

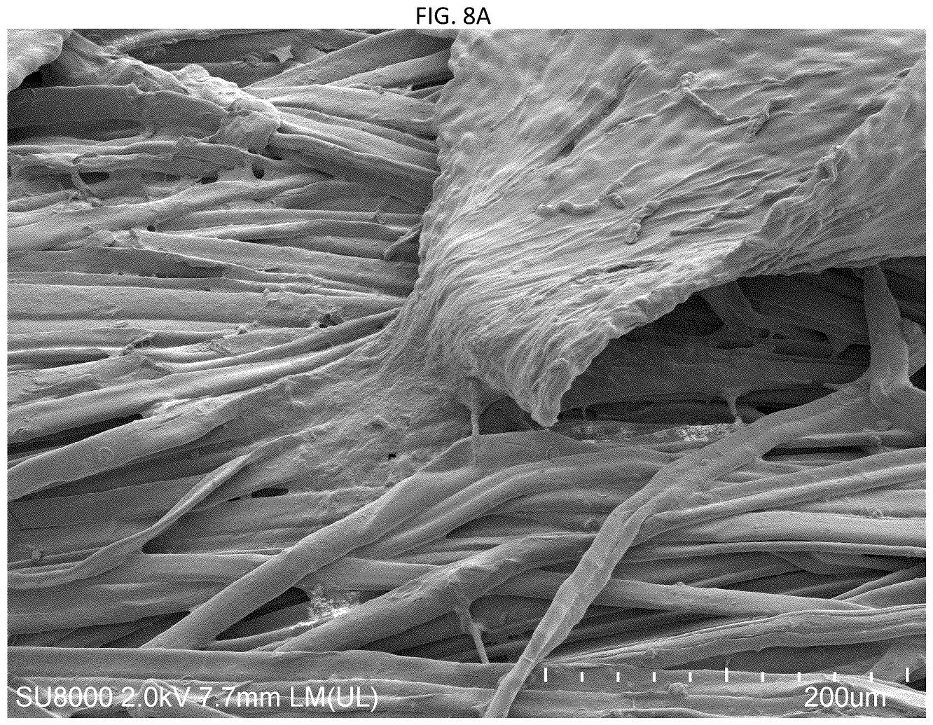

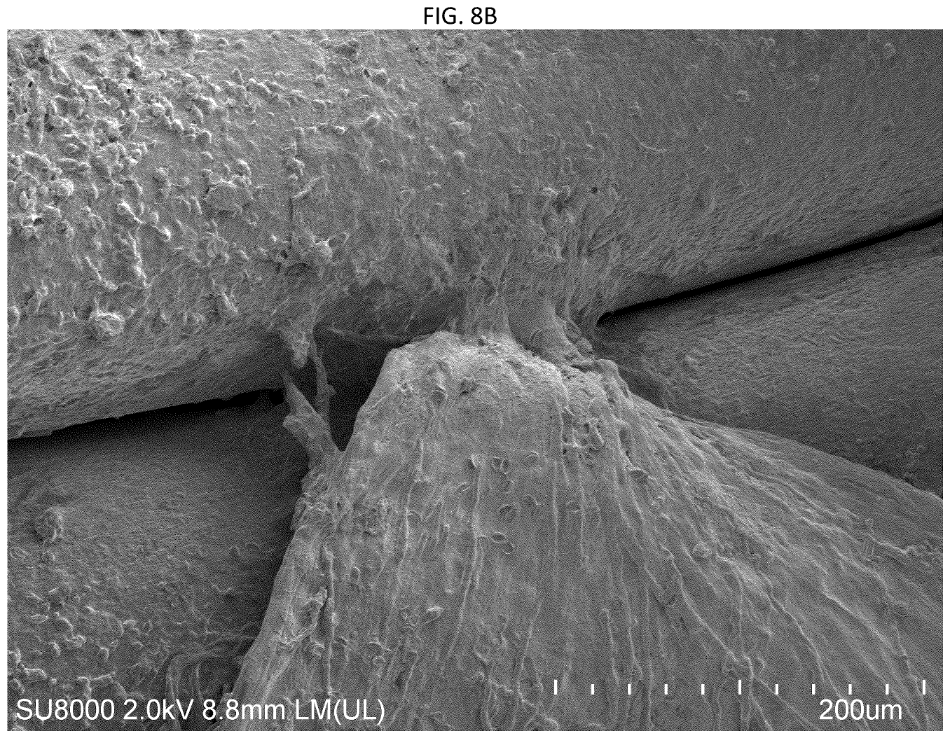

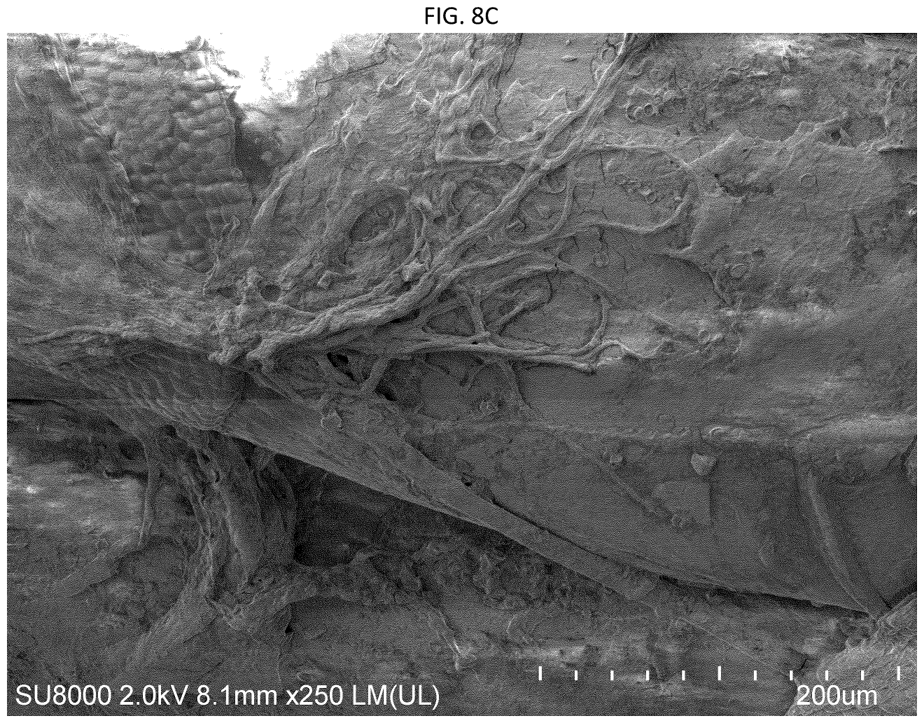

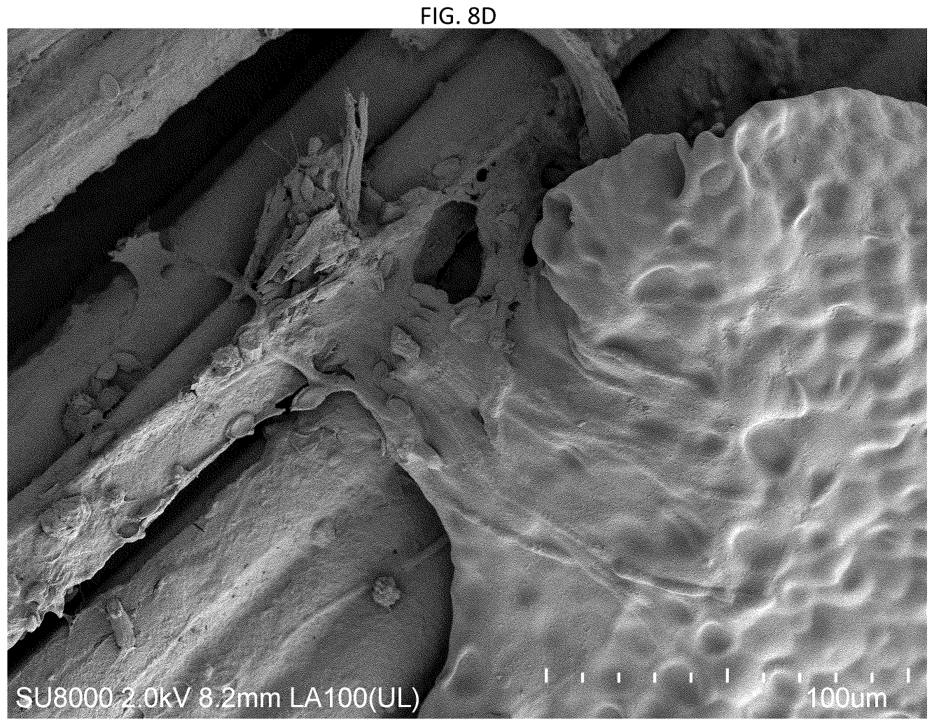

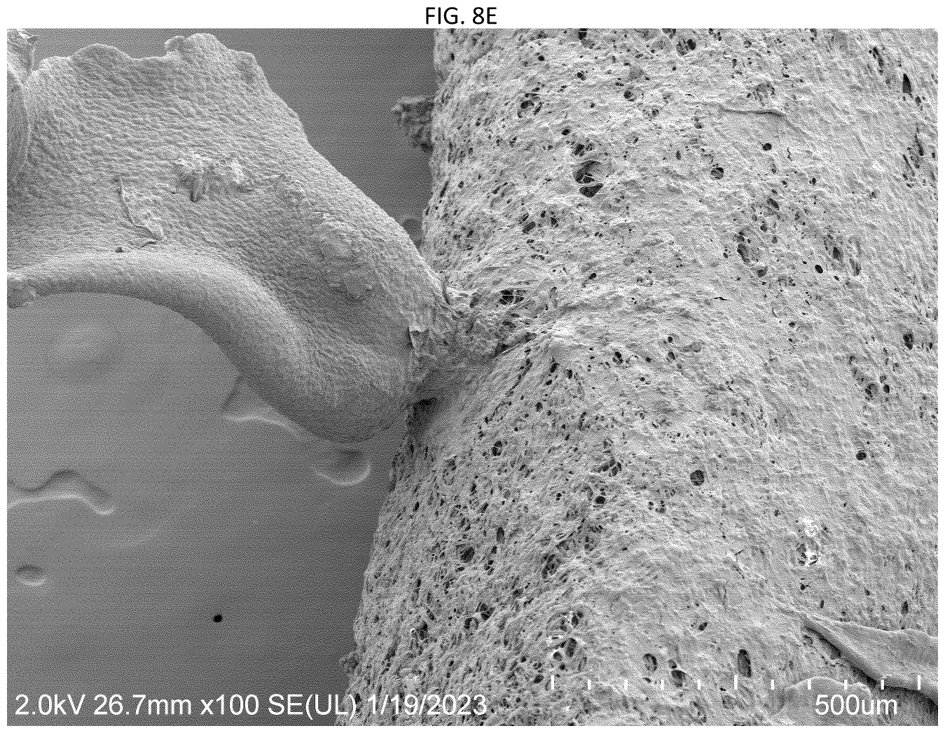

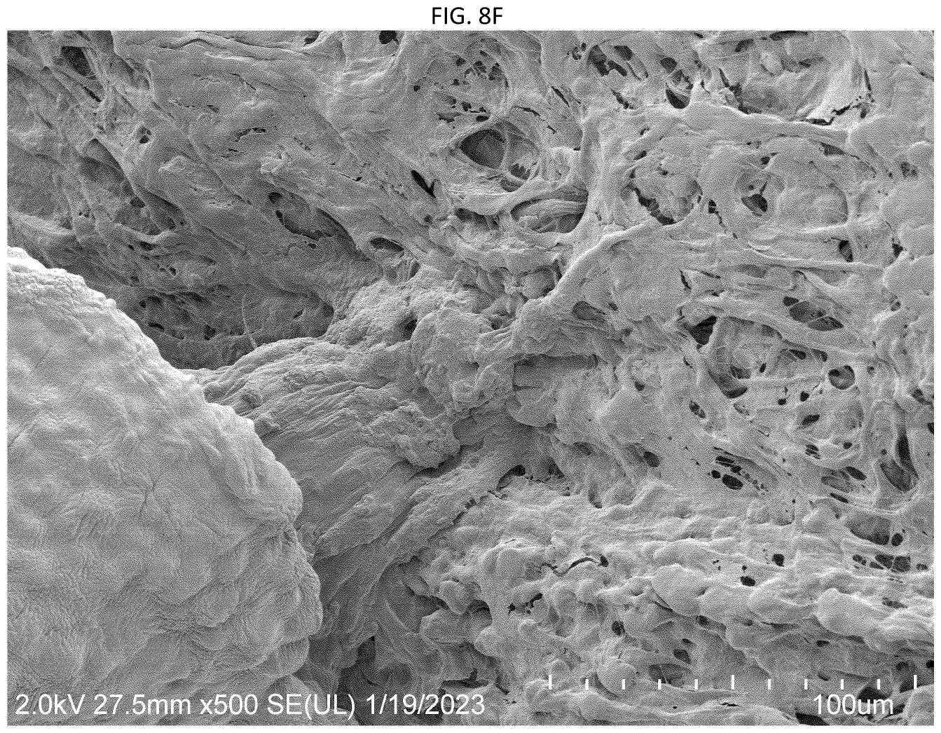

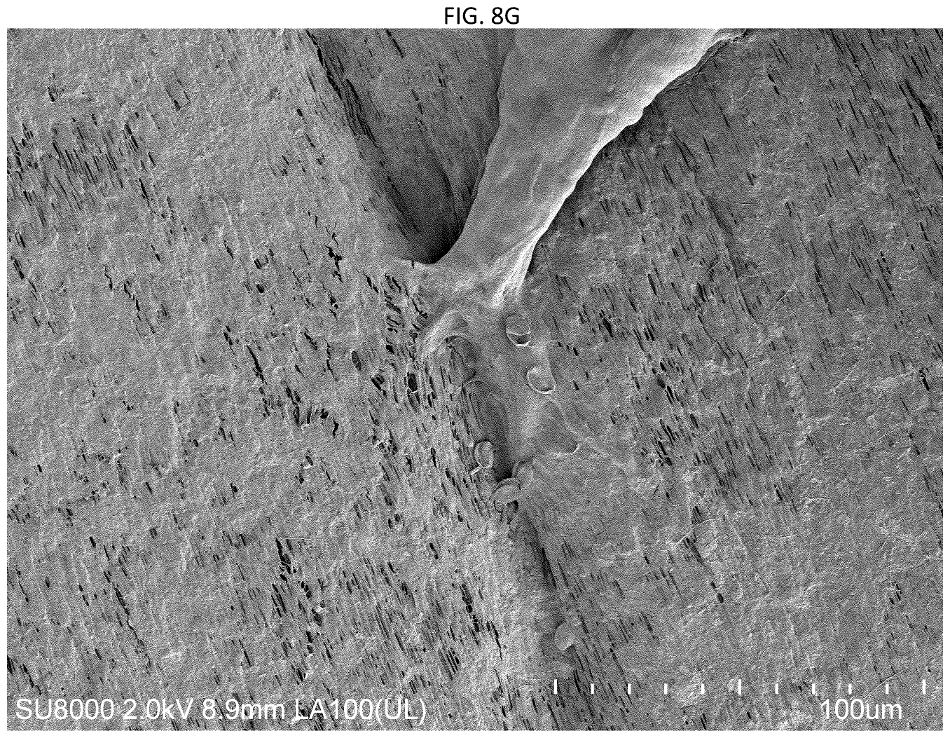

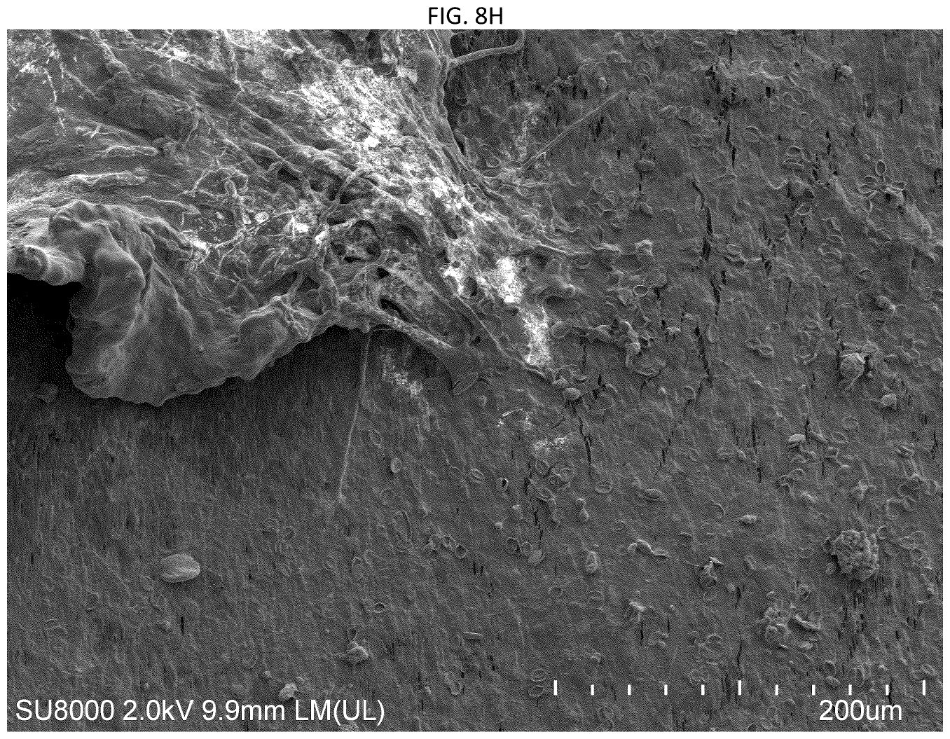

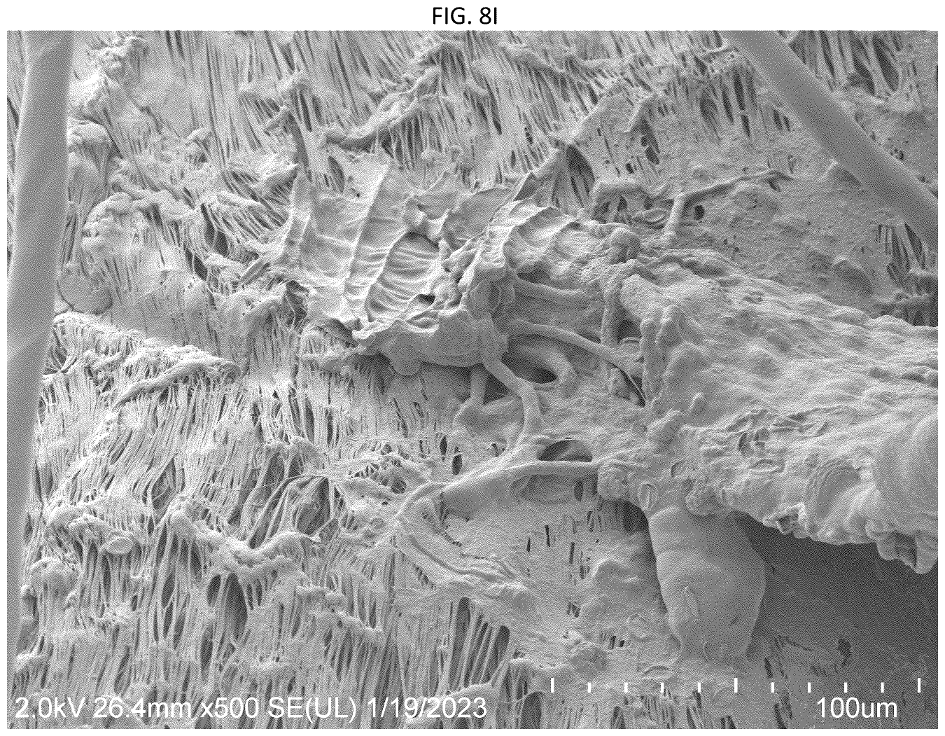

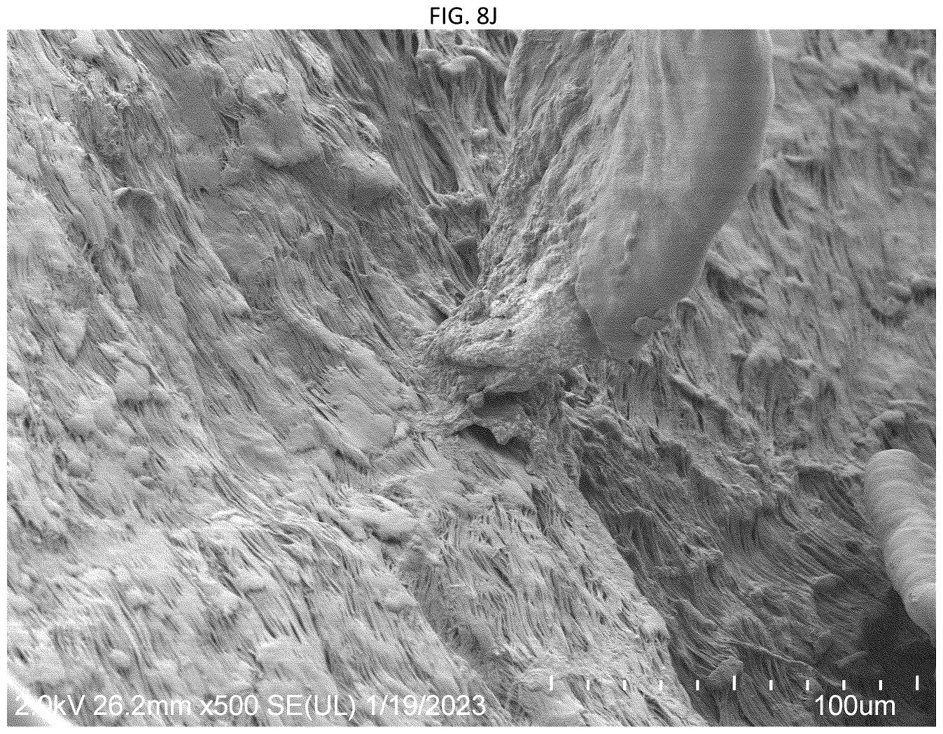

The substrates attached with small N. yezoensis blades were observed using Scanning Electron Microscope (SEM) to compare the holdfast attachment of blades on each of the different substrates (FIG. 8). Samples in salt water were prepared by cutting an appropriate length of the fiber in an area of interest using a razor blade (Ted Pella, Redding, CA, USA). Excess liquid was removed with a laboratory grade tissue, such as kimwipes (Kimberly-Clark, Irving, TX, USA). The smaller samples were mounted directly to an aluminum sample mount (Ted Pella, Redding, CA, USA) with double-sided acrylic adhesive tabs (SPI Supplies, West Chester, PA, USA) and air dried 24 hrs. Samples were then coated with a thin layer of platinum with a sputter coater (Cressington, Oxhey, Watford, UK) and imaged in a Hitachi SU8000 scanning electron microscope (Hitachi, Tokyo, Japan) at 2 kV. Alternatively, the smaller samples were rinsed and stabilized with 10% HILEM® ionic liquid (Hitachi, Tokyo, Japan) solution in ethanol for 15 minutes to preserve the “hydrated” state of the samples. Excess liquid was removed with a kimwipe (Kimberly-Clark, Irving, TX, USA). The samples were mounted directly to an aluminum sample mount (Ted Pella, Redding, CA, USA) with double-sided acrylic adhesive tabs (SPI Supplies, West Chester, PA, USA) [1,2]. The samples were transferred into the SEM without further drying or the addition of a conductive coating. The samples were imaged in a Hitachi SU8000 scanning electron microscope (Hitachi, Tokyo, Japan) at 2 kV.

The result of SEM observation shows that the blades on substrate A (FIGS. 8E and 8F), substrate B (FIGS. 8G and 8H) and substrate C (FIGS. 81 and 8J) all showed very good attachment on the substrate with incorporation into the substrate structure, particularly with incorporation into the micropores throughout the substrate. Substrates JP1 (FIG. 8A) and JP2 (FIG. 8B) both show good attachment. The blades on substrates CN (FIG. 8C) and KR (FIG. 8D) showed attachment to single filaments resulting in weak attachment.

### Example 4: Overall Evaluation of Substrate

Comprehensive evaluation of the different substrates based on their performance in the conchospores attachment (seeding), germling growth rate and yield was performed using the data described in the examples above. To directly evaluate and compare the overall performance of each substrate, the seeding density was multiplied by germling growth rate and then by yield to calculate the growth potential score of each substrate.

TABLE 2
Evaluation of overall growth potential of each substrate

| | Measured mean value | | | Growth |
|---|---|---|---|---|
| | Seeding | Growth | | potential |
| Seedstring | density | rate | Yield | score |
| JP1 | 7.30 | 5.39 | 3.07 | 120.80 |
| JP2 | 6.09 | 6.49 | 2.28 | 90.11 |
| CN | 10.72 | 1.89 | 1.42 | 28.77 |
| KR | 6.78 | 1.15 | 3.38 | 26.35 |
| Gore A | 6.49 | 10.43 | 4.10 | 277.53 |
| Gore B | 3.23 | 8.54 | 1.21 | 33.38 |
| Gore C | 25.20 | 3.99 | 9.38 | 943.14 |

An alternative means for evaluation of cultivation substrates also ranks the substrates according to their performance in seeding density, germling growth rate and yield. However, this evaluation scores substrates in each aspect by dividing that value by the highest value. Then, three scores were summed up to make the final ranking.

TABLE 3
Evaluation scores in seeding density, growth rate,
yield and overall score of each substrate

| | Evaluation score | | | |
|---|---|---|---|---|
| | Seeding | Growth | | Overall |
| Seedstring | density | rate | Yield | score |
| JP1 | 0.28 | 0.51 | 0.32 | 1.11 |
| JP2 | 0.24 | 0.62 | 0.24 | 1.10 |
| CN | 0.42 | 0.18 | 0.15 | 0.75 |
| KR | 0.26 | 0.11 | 0.35 | 0.72 |
| Gore A | 0.25 | 1 | 0.43 | 1.68 |
| Gore B | 0.12 | 0.81 | 0.12 | 1.05 |
| Gore C | 1 | 0.38 | 1 | 2.38 |

### Example 5: Mariculture Evaluation of Substrate

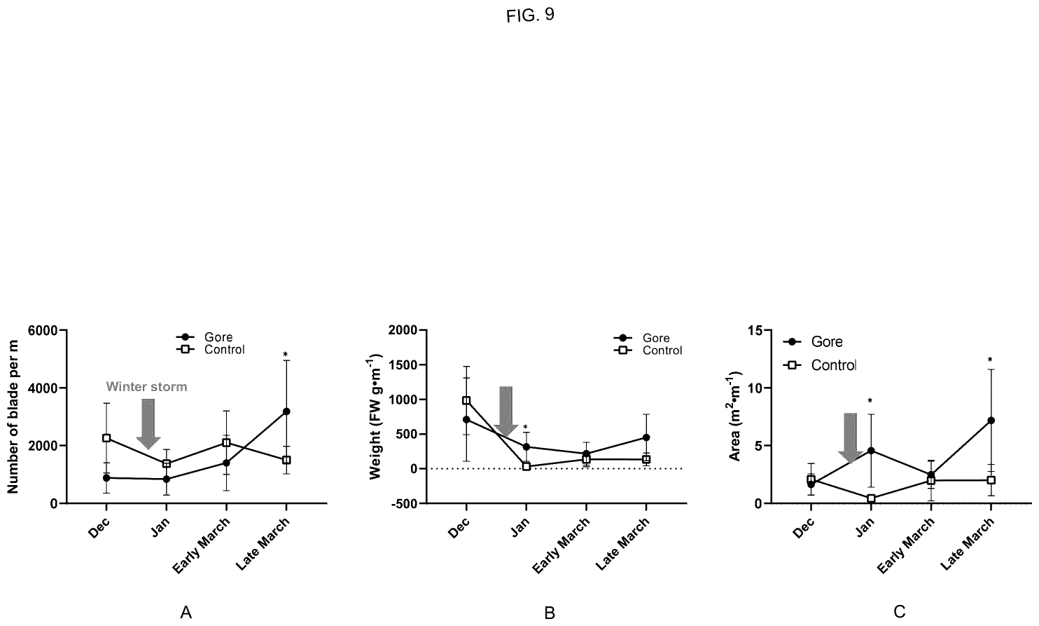

Two substrates were used to prepare cultivation nets for open water evaluation. The first cultivation net was prepared using the substrate KR, a commercial seedstring from Korea containing only polyamide (referenced as “control” in FIG. 9). The second cultivation net was prepared using the substrate C (containing 44% by weight of polyester and 56% by weight of microporous polytetrafluoroethylene) was made by twisting polyester around a microporous polytetrafluoroethylene core (Gore R6082) with a Ra surface roughness of 1.2 to form a non-porous:microporous expanded polymer substrate (labeled “Gore” in FIG. 9).

The two cultivation nets were seeded with conchospores in October and November and then the cultivation nets were outplanted to the farm site in Korea. Data was collected prior to the harvests of the Pyropia blades on the cultivation nets on Dec. 13, 2023, Jan. 30, 2024, Mar. 7, 2024 and Mar. 26, 2024. References to “early season” reflect the time closest to outplanting, such as the December and January harvest dates. References to “late season” reflect the time farther from outplanting, such as the early March and late March harvest dates.

Early in the growing season, a winter storm hit the site of the cultivation nets. As seen in FIG. 9, the storm conditions were sufficiently strong enough to remove most of the Pyropia blades from the control nets. FIG. 9B shows that, following the storm, the weight of Pyropia present on the control net was reduced to 0, but only reduced by half on the Gore net.

FIG. 9A shows the density of Pyropia (number of blades per m) on the cultivation nets at each designated harvest date. Not only did the density remain consistent before and after the winter storm, but the number of Pyropia blades continued to increase in March. The difference in number of blades per meter in late March was statistically significant, with the Gore net having approximately 3500 blades/m while the control net had less than 2000 blades/m. The increase in blade number late season (early March and late March) reflects a greater capture and engagement of archeospores by the Gore net in the early season generating new blade growth for harvest late season.

FIG. 9C shows the average area of the Pyropia blades on the cultivation net. This reflects the average of the size of the individual blades grown on the substrate. As shown in FIG. 9C, the area of the Pyropia blades was also significantly greater on the Gore net at both the Jan harvest (following the storm) and Late March harvests. The larger blade size in Late March again reflects a greater capture and engagement of archeospores by the Gore net in the early part of the season to generate new blade growth throughout the later half of the season.

Thus, the non-porous:microporous expanded polymer substrate of the present invention resulted in a seaweed cultivation medium providing Pyropia with an increased resistance to storm damage. The non-porous:microporous expanded polymer substrate of the present invention also resulted in a seaweed cultivation medium exhibiting an improved Pyropia harvest over the course of the growing season.

The foregoing examples illustrate various concepts described in association with the embodiments of this disclosure and are meant to be read collectively with those concepts.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention, provided they come within the scope of the appended claims and their equivalents.

1. A seaweed cultivation medium comprising:
a substrate composed of a microporous expanded polymer material and a non-porous material wherein the non-porous material is twisted around the microporous expanded polymer material,
wherein the substrate is exposed to a Pyropia-cultivation environment including seawater and Pyropia conchospores dispersed in the seawater to permit seeding onto the substrate, the Pyropia-cultivation environment including an aeration system and exposure to a seawater temperature of approximately 20 degrees Celsius and photosynthetically active radiation, and
wherein after 5 to 15 days of exposure to the Pyropia-cultivation environment, a surface of the submerged substrate supports a gametophyte density of 15-35 gametophytes per square millimeter.

2. A seaweed cultivation medium of claim 1 wherein the microporous expanded polymer material is expanded polyethylene (ePE) or expanded polytetrafluoroethylene (ePTFE).

3. A seaweed cultivation medium of claim 1 wherein the microporous expanded polymer material has a surface roughness (Ra) greater than 1.0.

4. A seaweed cultivation medium of claim 3 wherein the non-porous material is selected from the group consisting of polyester, polypropylene, polyamide, and polyethylene.

5. A seaweed cultivation medium of claim 3 wherein the microporous expanded polymer material exhibits no hairlike fibers protruding from the surface of the expanded polymer material

6. A seaweed cultivation medium of claim 3 wherein the substrate is exposed to the Pyropia-cultivation environment through periodic or continuous submersion of the substrate into the Pyropia-cultivation environment.

7. A seaweed cultivation medium of claim 6 wherein the substrate is exposed to the Pyropia cultivation environment less than 15 days.

8. A seaweed cultivation medium of claim 3, wherein the Pyropia conchospores are provided to the Pyropia-cultivation environment through oyster shells with conchocelis of Pyropia.

9. A seaweed cultivation medium of claim 8, wherein the Pyropia-cultivation environment comprises a ratio of about 2 oyster shells per liter of seawater.

10. A seaweed cultivation medium of claim 8 wherein the Pyropia-cultivation environment comprises a ratio of less than 2 oyster shells per liter of seawater.

11. A seaweed cultivation medium of claim 3 wherein the substrate has a growth potential value greater than or equal to 200, wherein the growth rate potential is determined by multiplying the average seeding density with the average germling growth rate and then by the average biomass yield for the substrate.

12. A seaweed cultivation medium of claim 3 wherein the substrate has an overall evaluation score greater than or equal to 1.5, wherein the overall evaluation score is determined by summing the (seeding density value/highest value) and the (germling growth rate/highest growth rate) and the (biomass yield/highest biomass yield).

13. A seaweed growth medium comprising:
a substrate composed of a microporous expanded polymer material having a surface roughness (Ra) greater than 1.0,
wherein the substrate is exposed to a Pyropia-cultivation environment including seawater and Pyropia conchospores dispersed in the seawater to permit seeding onto the substrate, and
wherein 30 days after seeding, the substrate supports juvenile Pyropia plants having an average blade size of 8-11 square millimeters.

14. A seaweed cultivation medium of claim 13 wherein the microporous expanded polymer material is expanded polyethylene (ePE) or expanded polytetrafluoroethylene (ePTFE).

15. The seaweed growth medium of claim 14, wherein the Pyropia exhibits an average growth rate of 0.25-0.35 mm²/day during the 30 days after seeding.

16. A seaweed growth medium comprising:
a substrate composed of a microporous expanded polymer material and a non-porous material wherein the non-porous material is twisted around the microporous expanded polymer material,
wherein the substrate is disposed in a Pyropia-cultivation environment including seawater and Pyropia spores dispersed in the seawater to permit seeding onto the submerged substrate, and
wherein 60 days after seeding, a surface of the submerged substrate supports an average Pyropia biomass yield of 6-10 mg/mm², wherein the surface seawater was removed from the Pyropia biomass prior to weighing.

17. A seaweed cultivation medium of claim 16 wherein the microporous expanded polymer material is expanded polyethylene (ePE) or expanded polytetrafluoroethylene (ePTFE).

18. A seaweed cultivation medium of claim 16 wherein the expanded polymer material has a surface roughness (Ra) greater than 1.0.

19. A seaweed cultivation medium of claim 16 wherein the non-porous material is selected from the group consisting of polyester, polypropylene, polyamide, and polyethylene.

20.-30. (canceled)

31. A seaweed cultivation medium comprising:
a substrate composed of a microporous expanded polymer material and a non-porous material wherein the non-porous material is twisted around the microporous expanded polymer material,
wherein the substrate is seeded with Pyropia conchospores,
wherein about three months after outplanting to an open water farm, the blade number and blade area exhibited by the Pyropia on the substrate are increased over the blade number and blade area of Pyropia grown on a cultivation medium comprising a substrate composed of polyamide.

32. The seaweed cultivation medium of claim 31 wherein the average Pyropia blade number about three months after outplanting is twice the average blade number of Pyropia grown on a cultivation medium comprising a substrate composed of polyamide.

33. The seaweed cultivation medium of claim 31 wherein about three months after outplanting, the average Pyropia blade number is three times the average blade number of Pyropia grown on a cultivation medium comprising a substrate composed of polyamide.

34. The seaweed cultivation medium of claim 31 wherein about three months after outplanting, the average blade area (m² per m) is twice the average blade area of Pyropia grown on a cultivation medium comprising a substrate composed of polyamide.

35. A seaweed cultivation medium of claim 31 wherein the microporous expanded polymer material is expanded polyethylene (ePE) or expanded polytetrafluoroethylene (ePTFE).

36. A seaweed cultivation medium having resistance to Pyropia loss from a storm condition, comprising:
a substrate composed of a microporous expanded polymer material and a non-porous material wherein the non-porous material is twisted around the microporous expanded polymer material,
wherein the substrate is seeded with Pyropia conchospores, and
wherein the weight (grams/meter) of Pyropia on the substrate is calculated at a first time period and at a second time period,
wherein the substrate is exposed to the storm condition, the storm condition being capable of removing seaweed from the substrate between the first time period and a second time period;
wherein difference in Pyropia weight between the first time period and the second time period is less than the difference in Pyropia weight between the first time period and the second time period for Pyropia grown on a substrate composed of polyamide.

37. A seaweed cultivation medium of claim 35 wherein the microporous expanded polymer material is expanded polyethylene (ePE) or expanded polytetrafluoroethylene (ePTFE).
